SEQUENCE LISTING (10 sequences)

SEQ ID NO: 1 - DNA, 20 bp, Synthetic construct
  source 1..20: mol_type = other DNA
caagggctgcatgactggct

SEQ ID NO: 2 - DNA, 22 bp, Synthetic construct
  source 1..22: mol_type = other DNA
gtctcctctgacttcaacagcg

SEQ ID NO: 3 - DNA, 22 bp, Synthetic construct
  source 1..22: mol_type = other DNA
accaccctgttgctgtagccaa

SEQ ID NO: 4 - DNA, 21 bp, Synthetic construct
  source 1..21: mol_type = other DNA
ccacttacctgaatgaccacc

SEQ ID NO: 5 - DNA, 20 bp, Synthetic construct
  source 1..20: mol_type = other DNA
gtggccttctttgagttcgg

SEQ ID NO: 6 - DNA, 20 bp, Synthetic construct
  source 1..20: mol_type = other DNA
gccgtacagttccacaaagg

SEQ ID NO: 7 - DNA, 20 bp, Synthetic construct
  source 1..20: mol_type = other DNA
ttccgactgaagagtgagcc

SEQ ID NO: 8 - DNA, 20 bp, Synthetic construct
  source 1..20: mol_type = other DNA
ccaagaaagctgcatcgaac

SEQ ID NO: 9 - DNA, 19 bp, Synthetic construct
  source 1..19: mol_type = other DNA
aactccacaaacccatccc

SEQ ID NO: 10 - DNA, 21 bp, Synthetic construct
  source 1..21: mol_type = other DNA
ccacttacctgaatgaccacc

(19) United States
(12) Patent Application Publication
(10) Pub. No.: US 2026/0124240 A1
(43) Pub. Date: May 7, 2026

(54) DRUG RESISTANT IMMUNE CELLS

(71) Applicant: The Regents of the University of California, Oakland, CA (US)

(72) Inventors: Dan S. Kaufman, La Jolla, CA (US); Davide Bernareggi, San Diego, CA (US); Benjamin H. Goldenson, Oakland, CA (US)

(21) Appl. No.: 19/117,254

(22) PCT Filed: Oct. 24, 2023
(86) PCT No.: PCT/US2023/077663

§ 371 Date: Mar. 31, 2025

Related U.S. Application Data
(60) Provisional application No. 63/380,698, filed on Oct. 24, 2022.

(51) Int. Cl. A61K 35/17; A61K 31/635; A61K 40/15; A61K 40/31; A61P 35/02; C07K 14/47; C12N 5/0783

(52) U.S. Cl. CPC A61K 35/17; A61K 31/635; A61K 40/15; A61K 40/31; A61P 35/02; C07K 14/4703; C07K 14/4747; C12N 5/0646; C12N 2510/00

(57) ABSTRACT

Compositions and method of prevention and treatment for a subject in need comprising drug-resistant natural killer (NK) cells, which are effective for treating cancer when administered in conjunction with cytotoxic therapies. A method of treatment comprising administering to a subject in need thereof an effective amount of modified NK cells and a cytotoxic therapy. A purified cell composition comprising modified NK cells. A pharmaceutical composition comprising an effective amount of G101V mutated NK cells and venetoclax.

### CROSS REFERENCE

This application claims the priority benefit of U.S. Provisional Application No. 63/380,698, filed Oct. 24, 2022, the entirety of which is hereby incorporated by reference herein in its entirety.

### GOVERNMENT SPONSORSHIP

This invention was made with government support under Grant No. U01CA217885 awarded by National Institutes of Health. The government has certain rights in the invention.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “24978-0854_Sequence Listing” was created on Oct. 23, 2023, and is 13000 bytes in size.

### TECHNICAL FIELD

The present invention relates generally to cellular therapies for treating cancer and other diseases.

### BACKGROUND

Acute myeloid leukemia (AML) is an often fatal malignancy with few good curative options. The incidence of AML increases with age, and older patients are typically less able to tolerate more aggressive therapies, typically “7+3” treatment that consists of cytarabine continuously for 7 days, along with infusions of an anthracycline (daunorubicin or idarubicin). Venetoclax in combination with hypomethylating agents (azacytidine or decitabine) is approved for treatment of older patients with AML who cannot tolerate this 7+3 regimen. While the venetoclax-based regimen can be effective to induce remissions, as noted, these patients will inevitably relapse.

NK cells have also been shown in clinical trials to be effective to induce remissions for relapsed or refractory AML. However, this treatment is also not typically curative. Additionally, NK cells cannot be given at the same time as cytotoxic therapies, such as venetoclax, as the treatment would kill off the NK cells. Thus, a treatment for more aggressively treating AML (or other malignancies) by administering venetoclax and NK cells is desirable.

### SUMMARY OF THE INVENTION

Disclosed herein are compositions and method of prevention and treatment for a subject in need comprising drug-resistant immune cells, such as natural killer (NK) cells, which may be effective for treating cancer when administered in conjunction with cytotoxic therapies. In embodiments, a method of treatment is provided, the method comprising administering to a subject in need thereof an effective amount of cytotoxic-resistant modified NK cells and a cytotoxic therapy. In some embodiments, the modified NK cells are BCL2 G101V mutated NK cells, and the cytotoxic therapy is venetoclax. In some embodiments, the modified NK cells are derived from induced pluripotent stem cell (iPSC)-derived immune cells.

In certain aspects, provided herein is a method of treating or preventing a disease or disorder in a subject in need thereof comprising administering to a subject in need thereof an effective amount of cytotoxic-resistant modified natural killer (NK) cells and a cytotoxic therapy. In some embodiments, the cytotoxic therapy is venetoclax. In some embodiments, the disease or disorder is cancer. In some the modified NK cells are BCL2 G101V mutated NK cells. In some embodiments, the BCL2 G101V mutated cells are effective to mediate a resistance to the cytotoxic therapy.

In some embodiments, the disease or disorder is cancer. In some embodiments, the cancer is acute myeloid leukemia (AML). In some embodiments, the AML is resistant or refractory to venetoclax.

In some embodiments, administering the effective amount of modified NK cells and the cytotoxic therapy is effective to treat the disease or disorder without inhibiting cell product activity. In some embodiments, administering the effective amount of modified NK cells with the cytotoxic therapy is effective to treat cancer without inhibiting cell product activity.

In embodiments, a purified cell composition comprising modified NK cells is provided. In embodiments, a purified cell composition comprising cytotoxic-resistant modified NK cells is provided. In some embodiments, the modified NK cells are BCL2 G101V mutated NK cells. In some embodiments, the modified NK cells comprise homozygous BCL2 G101V mutations. In some embodiments, the modified NK cells comprise heterozygous BCL2 G101V mutations. In some embodiments, the modified NK cells are induced pluripotent stem cell (iPSC)-derived natural killer cells. In some embodiments, the modified NK cells comprise homozygous inactivating mutations in a cytokine-inducible SH2-containing protein (CISH) gene. In some embodiments, the modified NK cells comprise a chimeric antigen receptor.

In embodiments, a pharmaceutical composition comprising an effective amount of BCL2 G101V mutated NK cells and venetoclax is provided. In some embodiments, the pharmaceutical composition is effective for treating cancer.

In another aspect, provided herein is a pharmaceutical composition comprising the purified cell composition of the present disclosure, and a pharmaceutically acceptable carrier. In some embodiments, the pharmaceutical composition further comprises venetoclax.

In yet another aspect, provided herein is a method of treating cancer in a subject in need thereof, comprising administering the pharmaceutical composition of the present disclosure to the subject. In some embodiments, the cancer is AML. In some embodiments, the cancer is resistant or refractory to a BCL2 inhibitor. In some embodiments, the BCL2 inhibitor is selected from the group consisting of venetoclax, navitoclax, and obatoclax. In specific embodiments, the BCL2 inhibitor is venetoclax.

### BRIEF DESCRIPTION OF THE DRAWINGS

FIG. 1: Mechanisms of venetoclax activity. In cancer cells, the anti-apoptotic protein BCL2 sequesters and blocks the function of BH3-pro-apoptotic proteins (e.g., BIM) and therefore prevents apoptosis. The BH3-mimetic compound venetoclax displaces and reactivates pro-apoptotic proteins bound to the BH3-binding groove of BCL2. Consequently, released pro-apoptotic proteins associate with the apoptotic effectors BAX and BAK and induce permeabilization of the mitochondrial outer membrane. Cytochrome c released from mitochondria then activates caspases and triggers cell death. (Figure from Mihalyova J. et al., Experimental Hematology (2018).

FIGS. 2A-2D: Rationale for development of venetoclax resistant NK cells. FIG. 2A. Venetoclax binds BCL2 which prevents the protein from its normal activity to inhibit the apoptotic pathway. Therefore, the tumor cells are killed by the drug. FIG. 2B. Tumor cells can develop mutations to make them become resistant to venetoclax. Mutated BCL2 no longer binds to venetoclax therefore cells causing the mutated tumor cells to expand and lead to disease relapse. FIG. 2C. Combining NK cell therapy with venetoclax therapy would normally lead to killing of the NK cells, due to the effects of venetoclax on the normal immune cells, making this NK cell treatment less effective. FIG. 2D. NK cells bearing the G101V mutation can survive when exposed to venetoclax. Therefore, this cell engineering enables persistence and improved activity of the engineered NK cells that can be given at the same time as venetoclax to improve their anti-tumor activity and prevent development of cancer relapse from venetoclax resistance.

FIG. 3: Generation of Bcl2 G101V mutation in iPSCs. Top panel. Schematic showing the G101V mutation in BCL2 gene. Bottom panel. Homology directed repair insertion/deletion showing homozygous mutation.

FIG. 4: BCL-2 mutated 1F6 iPSC-derived NK cells have a normal NK cell phenotype. Differentiated WT iPSC-derived NK and BCL2 mutated 1F6 iPSC-derived NK cells were analyzed by flow cytometry for CD56 and other typical NK cell surface receptors demonstrating a similar phenotype.

FIGS. 5A-5B: BCL2 G101V iPSC-derived NK cells are resistant to venetoclax. Dose-response curves for FIG. 5A. WT iPSC-derived NK and FIG. 5B. 1F6 venetoclax-resistant iPSC-derived NK cells treated with Venetoclax.

FIGS. 6A-6B: NK cell activation assays. NK cells have been stimulated with Molm13 wt or resistant to venetoclax (VX) cells with or without venetoclax 3 μM in the cell culture media. 1F6 iPSC-derived NK cells have better function in vitro when treated with Venetoclax. FIG. 6A. production of CD107a, TNFα, and IFNγ in response to Molm13 WT and Molm13 VX cells were measured. iPSC-derived NK cells and 1F6 iPSC-derived NK cells were stimulated with 1:1 ratio of target cells and stained for CD107a, TNFα, and IFNγ 4 hours later. FIG. 6B. WT iPSC-derived NK cells and 1F6 iPSC-derived NK cells were treated with 3 μM of Venetoclax 24 hours prior to running the assay. Representative of n=3 independent experiments.

FIGS. 7A-7D: BCL2 G101V iPSC-derived NK cells-mediated cytotoxicity is resistant to venetoclax treatment. NK cells are stimulated by Molm13 cells. FIG. 7A. WT iPSC-derived NK killing and 1F6 iPSC-derived NK cells killing FIG. 7B. against Molm13 WT cells was tested over an extended time course using the Incucyte real time imaging system at the indicated effector to target (E:T) ratio over 36 hours. Both the WT and 1F6-iPSC-derived NK cells show similar killing of the tumor cells, as indicated by decreasing values over time. FIG. 7C-7D. 1.5 M of venetoclax was added to the assay with (FIG. 7C) WT iPSC-derived NK cells and (FIG. 7D) 1F6 iPSC-derived NK cell-mediated killing against venetoclax-resistant Molm13 measured. These studies demonstrate that the WT-iPSC-derived NK cells fail to kill the venetoclax-resistant Molm13 cells in the presence of venetoclax. However, the 1F6 iPSC-derived NK cells demonstrate potent killing of venetoclax-resistant Molm13 cells, even in the presence of venetoclax. Each experiment was done in triplicate. Representative of n=2 independent experiments.

FIG. 8: BCL2 G101V iPSC-derived NK cells are resistant to venetoclax. Dose-response curves for WT-iPSC-derived NK and BCL2 G101V venetoclax-resistant iPSC-derived NK cells treated with Venetoclax.

FIGS. 9A-9F: Introduction of the BCL2 G101V mutation in iPSCs and differentiation of iPSCs-NK cells and Characterization of Engineered iPSCs. FIG. 9A. Schematic showing knock-in of the G101V mutation in the BCL2 gene via CRISPR/Cas9 homology directed repair. FIG. 9B. Diagram generated from Sanger sequencing confirming the homozygous knock-in of the BCL2 G101V DNA coding sequence change. FIG. 9C. Sanger sequencing of heterozygous BCL2^{WT/G101V} iPSCs. FIG. 9D. Sanger sequencing of homozygous BCL2G101V/G101V iPSCs. FIG. 9E. Flow cytometry for expression of TRA-1-81 and SSEA-4 on BCL2WT/G101V iPSCs. FIG. 9F. Flow cytometry for expression of TRA-1-81 and SSEA-4 on BCL2G101V/G101V iPSCs.

FIG. 10: Schematic of the differentiation process by which BCL2 G101V iPSCs undergo hematopoietic differentiation prior to further differentiation under NK cell specific conditions to generate BCL2 G101V iPSC-derived NK cells.

FIGS. 11A-11C: Differentiated WT iPSC-derived NK and BCL2 mutated iPSC-derived NK cells demonstrate typical NK cell surface receptor phenotypes. FIG. 11A. Flow cytometry for the indicated NK cell surface markers. Representative of n=3 independent experiments. FIG. 11B-C. WT (FIG. 11B) and BCL2 G101V iPSC-derived NK (FIG. 11C) cell cytotoxicity against WT MOLM13 cells.

FIGS. 12A-12H: BCL2 G101V iPSC-derived NK cells are resistant to venetoclax and anti-apoptotic BCL2 family member levels are unchanged. Dose response curves for WT iPSC-derived NK (FIG. 12A), Homozygous BCL2 G101V iPSC-derived NK cells (FIG. 12B), and Heterozygous BCL2 G101V iPSC-derived NK cells (FIG. 12C) treated with Venetoclax and assessed for viability at 72 hours post treatment. FIG. 12D. Dose response for WT iPSC-derived NK and homozygous BCL2 G101V iPSC-derived NK cells treated with dexamethasone and assessed for viability at 72 hours post treatment. Qualitative real-time PCR analysis of BCL2 (FIG. 12E), BCL-XL (FIG. 12F), and MCL1 (FIG. 12G) mRNA level for WT iPSC-derived NK and BCL2 G101V iPSC-derived NK cells vehicle or venetoclax treated. FIG. 12H. Immunoblots for BCL2, and BCL-XL protein levels with a β-actin loading control.

FIGS. 13A-13P: BCL2 G101V iPSC-derived NK cells maintain normal function when treated with Venetoclax. Production of CD107a and IFNγ in response to MOLM13-WT and venetoclax resistant MOLM13 (VX) cells. iPSC-derived NK cells and BCL2 G101V iPSC-derived NK cells were stimulated with 1:1 ratio of target cells and stained for CD107a and IFNγ 4 hours later. FIG. 13A-B. WT iPSC-derived NK cells and BCL2 G101V iPSC-derived NK cells were treated with vehicle 24 hours prior to the assay that quantifies the production of CD107a (FIG. 13A) and IFNγ (FIG. 13B). FIG. 13C-D. WT iPSC-derived NK cells and BCL2 G101V iPSC-derived NK cells were treated with 3 μM venetoclax 24 hours prior to the assay that quantifies the production of CD107a (FIG. 13C) and IFNγ (FIG. 13D). Representative of n=3 independent experiments. FIG. 13E-13P. Representative plots of flow Cytometric Analysis of iPSC-derived NK cell-mediated activity. WT or BCL2G101V/G101V iPSC-derived NK cells were stimulated with either PMA as a positive control, WT MOLM13 cells or venetoclax resistant MOLM13 cells and assessed for CD107a and interferon-γ expression by flow cytometry, without venetoclax treatment and with 1.5 μM venetoclax treatment. FIG. 13E. WT iPSC-derived NK cells were stimulated with PMA as a positive control without venetoclax treatment and assessed for CD107a expression by flow cytometry. FIG. 13F. WT iPSC-derived NK cells were stimulated with WT MOLM13 cells without venetoclax treatment and assessed for CD107a expression by flow cytometry. FIG. 13G. WT iPSC-derived NK cells were stimulated with venetoclax resistant MOLM13 cells without venetoclax treatment and assessed for CD107a expression by flow cytometry. FIG. 13H. BCL2G101V/G101V iPSC-derived NK cells were stimulated with PMA as a positive control without venetoclax treatment and assessed for CD107a expression by flow cytometry. FIG. 13I. BCL2G101V/G101V iPSC-derived NK cells were stimulated with WT MOLM13 cells without venetoclax treatment and assessed for CD107a expression by flow cytometry. FIG. 13J. BCL2G101V/G101V iPSC-derived NK cells were stimulated with venetoclax resistant MOLM13 cells without venetoclax treatment and assessed for CD107a expression by flow cytometry. FIG. 13K. WT iPSC-derived NK cells were stimulated with PMA as a positive control with 1.5 μM venetoclax treatment and assessed for CD107a expression by flow cytometry. FIG. 13L. WT iPSC-derived NK cells were stimulated with WT MOLM13 cells with 1.5 μM venetoclax treatment and assessed for CD107a expression by flow cytometry. FIG. 13M. WT iPSC-derived NK cells were stimulated with venetoclax resistant MOLM13 cells with 1.5 μM venetoclax treatment and assessed for CD107a expression by flow cytometry. FIG. 13N. BCL2^{G101V/G10IV} iPSC-derived NK cells were stimulated with PMA as a positive control with 1.5 μM venetoclax treatment and assessed for CD107a expression by flow cytometry. FIG. 13O. BCL2^{G101V/G10IV} iPSC-derived NK cells were stimulated with WT MOLM13 cells with 1.5 μM venetoclax treatment and assessed for CD107a expression by flow cytometry. FIG. 13P. BCL2^{G101V/G101V} iPSC-derived NK cells were stimulated with venetoclax resistant MOLM13 cells with 1.5 μM venetoclax treatment and assessed for CD107a expression by flow cytometry.

FIGS. 14A-14E: BCL2 G101V iPSC-derived NK cells are resistant to inhibition by venetoclax and maintain normal cytotoxicity during venetoclax treatment. FIG. 14A. WT iPSC-derived NK cell-mediated killing against WT-MOLM13 cells examined over 36 hours using the Incucyte real time imaging system at the indicated effector to target (E:T) ratio without addition of venetoclax. FIG. 14B. BCL2 G101V iPSC-derived NK cells mediated killing of WT-MOLM13 cells without addition of venetoclax. FIG. 14C. 1.5 M venetoclax was added during the assay and WT iPSC-derived NK killing against venetoclax-resistant MOLM13 cells was measured. FIG. 14D. 1.5 μM venetoclax was added during the assay and BCL2 G101V iPSC-derived NK killing against venetoclax-resistant MOLM13 cells was measured. FIG. 14E. Comparison of WT iPSC-derived NK cell to BCL2 G101V iPSC-derived NK cells for ability to kill venetoclax resistant MOLM13 cells in the presence of 1.5 μM venetoclax. Experiments were completed in triplicate.

FIG. 15: A schema of NK cell dosing regimen and venetoclax treatment plan. NSG mice were inoculated IV with 2.5×105 luciferase-expressing MOLM13 cells, and tumor engraftment was assessed by IVIS imaging 4 days later for baseline pre-treatment grouping. On day 4 after tumor inoculation, mice were either left untreated or treated with 1×107 WT iPSC-derived NK cells or BCL2 G101V iPSC-derived NK cells alone or in combination with venetoclax for 21 days. NK cells were supported by injection of IL-15 for the first week and IL-2 for three weeks with IVIS imaging done as indicated to monitor tumor progression.

FIGS. 16A-16C: BCL2 G101V iPSC-derived NK cells mediate improved tumor killing in combination with venetoclax in vivo. BCL2-G101V iPSC-derived NK cells effectively mediate improved tumor killing in combination with venetoclax against MOLM13 cells. FIG. 16A. Tumor burden was determined by BLI over the first 25 days. FIG. 16B. IVIS imaging flux mean±SEM for the mice. FIG. 16C. Statistics: two-tailed Student t-test, *p<0.05, **p<0.01. Kaplan-Meier curves demonstrating survival of the experimental groups. The median survival for untreated, WT iPSC-derived NK, venetoclax treated, BCL2 G101V iPSC-derived NK, WT iPSC-derived NK-venetoclax and BCL2 G101V iPSC-derived NK+venetoclax groups are 18.6, 46, 18.8, not reached, 22.8 and not reached days, respectively. Statistics: two-tailed Log-rank test.

FIGS. 17A-17G: Venetoclax resistant MOLM13 cells are more resistant to NK cell killing through down-regulation of Fas expression but are more susceptible to killing by BCL2 G101V iPSC-derived NK cells. FIG. 17A. In vitro cytotoxicity assay of WT iPSC-derived NK cell killing of WT and venetoclax resistant MOLM13 cells. FIG. 17B-C. Flow Cytometric Analysis of NK cell ligands for WT MOLM13 cells (FIG. 17B) and Venetoclax-resistant MOLM13 cells (FIG. 17C). Highlighted boxes demonstrate down-regulation of Fas expression on the venetoclax-resistant cells. Fas protein expression by flow cytometry in WT MOLM13 (FIG. 17D) and venetoclax resistant MOLM13 cells (red isotype control) (FIG. 17E). FIG. 17F. Fas RNA expression by qRT-PCR in WT and venetoclax resistant MOLM13 cells. Resistance to quizartinib did not lead to down regulation of Fas expression. FIG. 17G. 36-hour cytotoxicity assay using venetoclax resistant MOLM13 cells incubated with either venetoclax alone, venetoclax with WT iPSC-derived NK cells or venetoclax with BCL2G101V/G101V iPSC-derived NK cells. 1.5 μM venetoclax used for this assay.

### DETAILED DESCRIPTION

Disclosed herein are drug-resistant NK cells effective for treating cancer when administered in conjunction with cytotoxic therapies.

Treatment of AML remains challenging, particularly for patients unfit for intensive chemotherapy. Venetoclax, a BCL2 inhibitor, has significantly improved treatment outcomes for these AML patients unfit for induction chemotherapy. Our group has pioneered the production of NK cells from human induced pluripotent stem cells (iPSCs). iPSC-derived NK (iPSC-derived NK) cells effectively kill AML cells, similar to NK cells isolated from peripheral blood or cord blood. iPSCs provide an advantage of a stable platform for gene modifications and previous studies demonstrate we can engineer iPSCs to express or delete genes of interest to derive genetically modified iPSC-derived NK cells with improved anti-tumor activity. For example, deletion of the cytokine-inducible SH2-containing protein CIS (encoded by the gene CISH) leads to improved anti-AML activity (H. Zhu, et al., 2020), and iPSC-derived NK cells engineered to express novel NK cell-optimized chimeric antigen receptors (CARs) or a stabilized version of the Fc receptor CD16 leads to improved anti-tumor activity (Y. Li, et al., 2018; H. Zhu, et al., 2020). Based on the characterization of the BCL2 G101V mutation that provides resistance to the BCL2 inhibitor venetoclax in chronic lymphocytic leukemia, we investigated the effect of homozygous BCL2 G101V knock-in in iPSC-derived NK cells to test the hypothesis that these resistant cells could be combined with venetoclax to improve NK cell-mediated killing of AML cells.

Overexpression of BCL2 in some malignancies has been linked to increased resistance to chemotherapy. The mutation G101V in BCL2 has been shown to emerge in some patients with chronic lymphocytic leukemia who are treated with venetoclax (R. W. Birkinshaow et al, 2019). The G101V mutation mediates tumor cell resistance to venetoclax, therefore while tumor cells become resistant to the drug, the normal patient immune cells are still affected by venetoclax, resulting in cytopenias.

Cell-based cancer immunotherapy provides a key approach for cancer therapy. Chimeric antigen receptors (CAR)-expressing T cells are FDA approved for treatment of B cell malignancies and multiple myeloma. NK cells, T cells and macrophages are cell types now all used in clinical trials. These immune cell-based immunotherapies provide the ability to test combinatorial therapies using drugs like venetoclax in combination with NK cells, T cells or macrophages (these cells may or may not express CARs or other genome editing to improve their function) to treat tumors (solid tumors or hematological malignancies). However, without engineering resistance to venetoclax (or similar agent) into the immune cells, those cells would be inhibited by the drug activity and the cells would have poor survival and poor anti-tumor activity. The novelty of our approach is that we have engineered immune cells to be resistant to venetoclax to make the immune cells more resistant to keep the immune cells alive and more effective for use in combination with venetoclax.

To generate iPSC-derived NK cells with an improved ability to tolerate exposure to venetoclax, we employed CRISPR-Cas9 technology to knock-in the BCL2 G101V mutation (BCL2G101V) in iPSCs. iPSCs homozygous for BCL2G101V were selected and differentiated to NK cells. BCL2G101V iPSC-derived NK cells were 94-fold more resistant to venetoclax compared to wildtype iPSC-derived NK cells (Panel A). Analysis of cell surface proteins showed that both sets of iPSC-derived NK cells had a typical NK cell phenotype with no differences in expression of receptors analyzed. There was no difference seen in cytotoxicity against K562 tumor cells between BCL2G101V iPSC-derived NK cells and WT iPSC-derived NK cells.

Additional functional analyses demonstrated that activity of the BCL2^{G101V} iPSC NK cells was preserved upon exposure to venetoclax. CD107a expression on WT iPSCNK cells stimulated by MOLM13 AML tumors cells was reduced nearly 3-fold upon addition of venetoclax. In contrast, activation of BCL2 iPSC-derived NK cells was not significantly affected by addition of venetoclax. This resistance to venetoclax was confirmed in longer term 36-hour Incucyte cytotoxicity assays where BCL2^{G101V} iPSC-derived NK cells killed more than 10-fold the number of tumor cells than the WT-iPSC-derived NK cells (Panel B).

Intriguingly, BCL2^{G101V} iPSC-derived NK cells also demonstrated improved cytotoxicity against MOLM13 cells resistant to venetoclax. Further analyses into the mechanism of this property of BCL2^{G101V} iPSC-derived NK cells is in progress. In vivo studies testing the BCL2 G101V iPSC-derived NK cells in combination with venetoclax against WT and venetoclax-resistant MOLM13 of iPSC-derived NK cells in mouse xenograft models are also ongoing. Together our results demonstrate that iPSC-derived NK cells can be engineered to generate venetoclax-resistant cells to be used in combination with venetoclax therapy to improve treatment of AML. Furthermore, this work demonstrates that novel drug resistance mechanisms can be developed by genome engineering of iPSC-derived NK cells as a new strategy to produce improved cell products for “off-the-shelf” therapy.

Unless defined otherwise, all technical and scientific terms used herein have the same meanings as commonly understood by one of ordinary skill in the art to which the present disclosure belongs. Any materials and methods similar or equivalent to those described herein can be used to practice the present invention. The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (MJ. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and CC. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (CA. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al, eds., J. B. Lippincott Company, 1993). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, an engineered immune cell, a pharmaceutical composition, and/or a method that “comprises” a list of elements (e.g., components, features, or steps) is not necessarily limited to only those elements (or components or steps), but may include other elements (or components or steps) not expressly listed or inherent to the engineered immune cell, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the terms “a” and “an” as used herein refer to “one or more” of the enumerated components unless otherwise indicated. The use of the alternative (e.g., “or”) should be understood to mean either one, both, or any combination thereof of the alternatives.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment.

It will be further understood that there are a number of values disclosed therein, and that each value is also herein disclosed as “about” that particular value in addition to the value itself. In embodiments, “about” can be used to mean, for example, a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In various embodiments, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

All publications, patents, and patent applications mentioned in this specification are herein incorporated by reference to the same extent as if each individual publication, patent, or patent application was specifically and individually indicated to be incorporated by reference.

In some embodiments, the engineered immune cell is an induced pluripotent stem cell (iPSC)-derived immune cell. In some embodiments, the engineered immune cell is a peripheral blood (PB)-derived immune cell. In some embodiments, the engineered immune cell is a cord blood (CB)-derived immune cell.

As used herein, “induced pluripotent stem cell” or “iPSC cell” or “iPSCs” are used to refer to cells, derived from somatic cells, that have been reprogrammed back to an pluripotent state that are capable of proliferation, selectable differentiation, and maturation.

As used herein, “peripheral blood” or “peripheral blood cell” is used to refer to cells that originate from circulating blood and comprise hematopoietic stem cells that are capable of proliferation, selectable differentiation, and maturation.

As used herein, “cord blood cell” is used to refer to cells that originate from the umbilical cord and placenta and comprise hematopoietic stem cells that are capable of proliferation, selectable differentiation, and maturation.

In some embodiments, the engineered immune cell is a NK cell.

As used herein, and unless otherwise specified, a “natural killer cell” or “NK cell” is used to refer to cells that are cytotoxic lymphocytes that constitute a major component of the innate immune system. In humans a natural killer cell usually expresses the surface markers CD16 (FCyRIII) and CD56. NK cells are cytotoxic; small granules in cytoplasm that contain special proteins such as perforin and proteases known as granzymes. NK cells provide rapid responses to virally infected cells and respond to transformed cells. Upon release in close proximity to a cell slated for killing, perforin forms pores in the cell membrane of the target cell through which the granzymes and associated molecules can enter, inducing apoptosis. Thus, NK cells may act as effectors of lymphocyte population in anti-tumor and anti-infection immunity.

Typically, immune cells detect peptides from pathogens presented by Major Histocompatibility Complex (MHC) molecules on the surface of infected cells, triggering cytokine release, causing lysis or apoptosis. NK cells are unique, however, as they have the ability to recognize stressed cells regardless of whether peptides from pathogens are present on MHC molecules. They were named “natural killers” because of the initial notion that they do not require prior activation in order to kill a target. NK cells are large granular lymphocytes (LGL) and are known to differentiate and mature in the bone marrow from where they then enter into the circulation.

As used herein, “engineered” or “genetically modified” or “transformed” are used interchangeably, wherein a cell has been manipulated by means of molecular reprogramming of a genomic sequence (e.g. by insertion, deletion, or substitution). Said cells include the primary transformed cell and progeny derived therefrom without regard to the number of passages. Progeny may not be completely identical in nucleic acid content to a parent cell and may contain mutations. Mutant progeny that have the same function or biological activity as screened or selected for in the originally transformed cell are included herein.

### Drug Resistant Immune Cells

In one aspect, provided herein are immune cells (e.g., NK cells, T and B lymphocytes, macrophages, etc.) modified to be resistant to a BCL2 inhibitor. In some embodiments, the immune cells (e.g., NK cells, T and B lymphocytes, macrophages, etc.) are modified to be resistant to a BH3-mimetic that inhibits BCL2 from binding BAX or BAK. In some embodiments, the BCL2 inhibitor is selected from the group consisting of venetoclax, navitoclax, and obatoclax. In some embodiments, the BCL2 inhibitor is venetoclax.

In some embodiments, the immune cell comprises homozygous BCL2 G101V mutations. In some embodiments, the immune cell comprises heterozygous BCL2 G101V mutations. In some embodiments, the immune cell is autologous or allogeneic to a subject receiving administration of the NK cells. In some embodiments, the immune cell is allogeneic, relative to the subject. In other embodiments, the immune cell is autologous, relative to the subject.

The immune cells of the disclosure may be a purified cell population. As used herein, a composition containing a “purified cell population” or “purified cell composition” means that at least 30%, 50%, 60%, typically at least 70%, and more preferably 80%, 90%, 95%, 98%, 99%, or more of the cells in the composition are of a similarly identified cell phenotype.

The immune cells of the disclosure may be an isolated cell population. As used herein, the term “isolated” means material that is substantially or essentially free from components that normally accompany it in its native state. In particular embodiments, the term “obtained” or “derived” is used synonymously with isolated.

The immune cells described herein can be modified using methods known in the art. The various gene editing systems described herein may be used to modify the immune cell to delete, inactivate, reduce expression, or otherwise inhibit function of a target gene or a target gene product.

In an aspect, the disclosure provides a cellular culture comprising NK cells. In embodiments, the NK cells have been produced from induced pluripotent stem cells (iPSCs) (hereinafter “iPSC-derived NK cells”). In embodiments, the NK cells have been produced from peripheral blood cells or cord blood cells. In embodiments, the cells are human cells.

Methods for making iPSC-derived NK cells may be obtained by differentiating iPSCs using known methods. In certain embodiments, iPSC-derived NK cells are made using embryoid bodies. Exemplary methods are provided in US 2013/0287751 A1 and Zhu et al. Methods Mol. Biol. 2048:107-119 (2019). In another variation, hESC-NK cells are made using single cell differentiation, as described, for example, in Woll et al. Blood 113:6094-6101 (2009). In certain embodiments, iPSC-derived NK cells are made by differentiating single cells. Exemplary methods are provided in US 2021/0024891 A1 and US 2016/0097035 A1. hESC-/iPSC-derived NK cells may be phenotyped by measuring surface marker expression such as CD16, NKG2D, NKp44, NKp46, TRAIL, FasL, or combinations thereof. Killing activity of hESC-/iPSC-derived NK cells may be assessed. Functional assays to assess differentiation into NK cells include direct cytolytic activity tumor cells (such as killing of K562 cells), Caspase-3/7 flow cytometry assay, immunological assays for cytotoxic granule or cytokine release, or antitumor activity in vivo xenograft models. See, e.g., Woll et al. Blood 113:6094-6101 (2009); Hermanson et al. in Hematopoietic differentiation of human pluripotent stem cells. 69-19 (2015).

In some embodiments, the NK cells are CD45+/CD56+ double-positive. In some embodiments, the immune cell is CD45+. In some embodiments, the immune cell is CD56+. In some embodiments the immune cell is CD45+, CD56+, or CD45+/CD56+. Further illustrative methods for making and using engineered cells are provided in U.S. Pat. Appl. Pub. Nos. US 2018/0298101 A1, US 2021/0230548 A1, and US 2021/0145883 A1; and Int'l. Pat. Appl. Pub. Nos. WO 2018/075664 A1 (corresponding to U.S. patent application Ser. No. 17/481,404) and WO 2020/113029 A2 (corresponding to U.S. patent application Ser. No. 17/309,408), the disclosures of which are incorporated by reference herein in their entireties.

Disclosed herein are methods of making a NK cell culture. In certain aspects, provided herein is a purified cell composition comprising cytotoxic-resistant modified NK cells. In certain embodiments, the NK cells are resistant to a BCL2 inhibitor (e.g., venetoclax). In some embodiments, the NK cells comprise homozygous BCL2 G101V mutations. In some embodiments, the NK cells comprise heterozygous BCL2 G101V mutations. In some embodiments, the NK cells are autologous or allogeneic to a subject receiving administration of the NK cells. In some embodiments, the NK cells are allogeneic, relative to the subject. In other embodiments, the NK cells are autologous, relative to the subject. In certain embodiments, the NK cell is derived from a pluripotent cell (e.g., an embryonic stem cell or an induced pluripotent stem cell). In some embodiments, the NK cells are induced pluripotent stem cell (iPSC)-derived natural killer cells.

Genome editing tools such as the clustered regularly interspaced short palindromic repeats (CRISPR) system may be used to genetically modify cells. These and other well-known and new techniques, such as TALEN and Zinc Finger Nucleases, for generating NK cells of the present disclosure are contemplated by the present invention.

The NK cell may be genetically modified to comprise a polynucleotide encoding a CAR. Known vectors may be used to deliver a polynucleotide, which may be stably maintained by the cell and/or integrated into the genome of the cell. In some embodiments, the polynucleotide is knocked-in to the genome of a cell. In some embodiments, the polynucleotide is knocked into a safe harbor locus (e.g., AAVS1). In some embodiments, the polynucleotide is knocked into an essential gene (see, e.g., WO 2021/22615).

The NK cells described herein can be modified using methods known in the art. The various gene editing systems described herein may be used to modify the NK cell to delete, inactivate, reduce expression, or otherwise inhibit function of a target gene or a target gene product.

In some embodiments, the NK cell is CISH^{+/+}. In some embodiments, the NK cell comprises homozygous inactivating mutations in a cytokine-inducible SH2-containing protein (CISH) gene. In some embodiments, the NK cell comprises heterozygous inactivating mutations in a cytokine-inducible SH2-containing protein (CISH) gene. In some embodiments, the NK cell is CISH^{-/-}. In some embodiments, the NK cell is CISH^{+/-}. In some embodiments, the cell does not comprise homozygous inactivating mutations of the cytokine-inducible SH2-containing protein ((ISH) genes (e.g., is CISH^{+/+}). In some embodiments, the NK cell comprises homozygous inactivating mutations of the transforming growth factor beta receptor 2 (TGFBR2) genes (e.g., is TGFBR2^{-/-}). In some embodiments, the NK cell does not comprise homozygous inactivating mutations of the transforming growth factor beta receptor 2 (TGFBR2) genes (e.g., is TGFBR2^{+/+}). In some embodiments, the NK cell comprises homozygous inactivating mutations of the beta-2-microglobulin (B2M) genes (e.g., is B2M^{-/-}). In some embodiments, the NK cell comprises homozygous inactivating mutations of the class II major histocompatibility complex transactivator ((IITA) genes (e.g., is CIITA^{-/-}). In some embodiments, the NK cell comprises homozygous inactivating mutations of the regulatory factor X (RFX) genes (e.g., is RFX^{-/-}).

The term “nucleic acid” or “polynucleotide”, includes DNA and RNA such as genomic DNA, cDNA and mRNA, or combinations thereof. The nucleic acid may comprise, in addition to the sequence enabling the genetic modifications of the disclosure, further sequences such as those required for the transcription and/or translation of the nucleic acid enabling said genetic modifications. This may include a promoter, enhancer, transcription and/or translation initiation and/or termination sequences, selection markers, sequences protecting or directing the RNA and/or enabling the genetic modifications within the cell. The selection and combination of these sequences is within the knowledge of the person skilled in the art and may be selected in accordance with the cell the nucleic acid is intended for.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell, in order to confirm the presence of the recombinant DNA sequence in the host cell, or confirm effect of genomic modulation, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or other assays.

Generally, techniques for differentiating an induced pluripotent cell involve modulation of specific cellular pathways, either directly or indirectly, using polynucleotide-, polypeptide- and/or small molecule-based approaches. The developmental potency of a cell may be modulated, for example, by contacting a cell with one or more modulators. “Contacting”, as used herein, can involve culturing cells in the presence of one or more factors (such as, for example, small molecules, proteins, peptides, etc.). In some embodiments, a cell is contacted with one or more agents to induce cell differentiation. Such contact, may occur for example, by introducing the one or more agents to the cell during in vitro culture. Thus, contact may occur by introducing the one or more agents to the cell in a nutrient cell culture medium. The cell may be maintained in the culture medium comprising one or more agents for a period sufficient for the cell to achieve the differentiation phenotype that is desired.

As used herein, “differentiate” or “differentiated” are used to refer to the process and conditions by which immature (unspecialized) cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells. The process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

Differentiation marker gene(s) may be monitored to gauge a cells state of differentiation. As used herein, the term “differentiation marker gene,” or “differentiation gene,” refers to genes whose expression are indicative of cell differentiation occurring within a cell, such as a pluripotent cell. “Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate,” or “maintain,” refers to the sustaining, propagating (growing) and/or differentiating of cells outside of tissue or the body, for example in a sterile plastic (or coated plastic) cell culture dish or flask. “Cultivation,” or “maintaining,” may utilize a culture medium as a source of nutrients, hormones and/or other factors helpful to propagate and/or sustain the cells.

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. As such, the term “pluripotent stem cell”, as used herein, refers to a subset of undifferentiated cells that are capable of giving rise to hematopoietic stem and progenitor cells via hematopoietic transition.

### Methods of Manufacture

In an aspect, the disclosure provides methods of making NK cells comprising providing a pluripotent cell, such as an induced pluripotent stem cell (iPSC) or embryonic stem cell (ESC), and differentiating the pluripotent cell into an NK cell. In certain embodiments, the pluripotent cell comprises homozygous BCL2 G101V mutations. In some embodiments, the pluripotent cell comprises heterozygous BCL2 G101V mutations.

Genome editing tools such as the clustered regularly interspaced short palindromic repeats (CRISPR) system may be used to genetically modify cells. These and other well-known and new techniques, such as TALEN and Zinc Finger Nucleases, for generating NK cells of the present disclosure are contemplated by the present invention.

The pluripotent cell may be genetically modified to comprise a polynucleotide encoding a CAR prior to or after differentiation. Known vectors may be used to deliver a polynucleotide, which may be stably maintained by the cell and/or integrated into the genome of the cell. In some embodiments, the polynucleotide is knocked-in to the genome of a cell. In some embodiments, the polynucleotide is knocked into a safe harbor locus (e.g., AAVS1). In some embodiments, the polynucleotide is knocked into an essential gene (see, e.g., WO 2021/22615).

The pluripotent cells described herein can be modified using methods known in the art. The various gene editing systems described herein may be used to modify the pluripotent cell to delete, inactivate, reduce expression, or otherwise inhibit function of a target gene or a target gene product.

In some embodiments, the pluripotent cell is CISH^{+/+}. In some embodiments, the pluripotent cell comprises homozygous inactivating mutations in a cytokine-inducible SH2-containing protein (CISH) gene. In some embodiments, the pluripotent cell comprises heterozygous inactivating mutations in a cytokine-inducible SH2-containing protein (CISH) gene. In some embodiments, the pluripotent cell is CISH^{-/-}. In some embodiments, the pluripotent cell is CISH^{+/-}. In some embodiments, the pluripotent cell does not comprise homozygous inactivating mutations of the cytokine-inducible SH2-containing protein (CISH) genes (e.g., is CISH^{+/+}). In some embodiments, the pluripotent cell comprises homozygous inactivating mutations of the transforming growth factor beta receptor 2 (TGFBR2) genes (e.g., is TGFBR2^{-/-}). In some embodiments, the pluripotent cell does not comprise homozygous inactivating mutations of the transforming growth factor beta receptor 2 (TGFBR2) genes (e.g., is TGFBR2^{+/+}). In some embodiments, the pluripotent cell comprises homozygous inactivating mutations of the beta-2-microglobulin (B2M) genes (e.g., is B2M^{-/-}). In some embodiments, the pluripotent cell comprises homozygous inactivating mutations of the class II major histocompatibility complex transactivator (CIITA) genes (e.g., is CIITA^{-/-}). In some embodiments, the pluripotent cell comprises homozygous inactivating mutations of the regulatory factor X (RFX) genes (e.g., is RFX^{-/-}).

### Pharmaceutical Compositions

In an aspect, the disclosure provides a pharmaceutical composition comprising the engineered immune cell of the disclosure and one or more pharmaceutically acceptable excipients or diluents. In some embodiments, the pharmaceutical composition further comprises a BCL2 inhibitor (e.g., venetoclax).

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable diluent or excipient” or “pharmaceutically acceptable carrier” refers to an excipient, diluent, preservative, solubilizer, emulsifier, adjuvant, and/or vehicle with which an NK cell of the disclosure, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable diluent or excipient” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

Formulations of a pharmaceutical composition suitable for administration typically generally comprise the active ingredient combined with a pharmaceutically acceptable diluents or excipients, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Formulations may also include aqueous solutions which may contain excipients such as salts, carbohydrates and buffering agents or sterile, pyrogen-free, water. Exemplary administration forms may include solutions or suspensions in sterile aqueous solutions, for example, aqueous propylene glycol or dextrose solutions. Such dosage forms can be suitably buffered, if desired.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, and/or aromatic substances and the like which do not deleteriously interact with the formulation. In some embodiments, the pharmaceutical composition comprises said NK cells in combination with other therapeutically active agents.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

### Methods of Use

In an aspect, the disclosure provides a method of treating or preventing a disease or disorder in a subject in need thereof, comprising administering the immune cells of the disclosure or the pharmaceutical composition of the disclosure to the subject. In some embodiments, the disease or disorder is a malignancy (e.g., cancer). In some embodiments, the disease or disorder is cancer. In some embodiments, the cancer is acute myeloid leukemia (AML). In some embodiments, the AML is resistant or refractory to venetoclax.

In some embodiments, administering comprises administering a therapeutically effective amount to a subject.

In another aspect, provided herein is method of treating cancer in a subject in need thereof, comprising administering the pharmaceutical composition of the present disclosure. In some embodiments, the cancer is AML. In some embodiments, the cancer is resistant or refractory to a BCL2 inhibitor. In some embodiments, the BCL2 inhibitor is selected from the group consisting of venetoclax, navitoclax, and obatoclax. In some embodiments, the BCL2 inhibitor is venetoclax.

As used herein, the term “amount” refers to “an amount effective” or “an effective amount” of a cell to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. In some embodiments, the engineered immune cell or pharmaceutical composition comprising said engineered immune cell of the disclosure is administered in a prophylactically effective amount.

The immune cells or pharmaceutical compositions of the disclosure may be administered in a number of ways depending upon whether local or systemic treatment is desired.

The NK cells, or pharmaceutical compositions thereof, are typically suitable for parenteral administration, wherein administration includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intranasal, intratracheal, intraarterial, intrathecal, intraventricular, intraurethral, intracranial, intratumoral, intraocular, intradermal, intrasynovial injection or infusions, intra-tumoral; and kidney dialytic infusion techniques. In some embodiments, the immune cells, or pharmaceutical compositions of the present disclosure comprise intravenous administration. In some embodiments, the immune cells, or pharmaceutical compositions of the present disclosure comprise intra-tumoral administration. In some embodiments, the immune cells, or pharmaceutical compositions are administered to a patient in a similar fashion to previous clinical work with immune cell-based therapies using unmodified peripheral blood immune, or NK cells.

In some embodiments, the engineered immune cell or pharmaceutical composition comprising said immune cells of the disclosure are administered in combination with a combination partner. The term “combination” refers to either a fixed combination in one dosage unit form, or a kit of parts for the combined administration where the immune cells, or pharmaceutical composition comprising said NK cells of the disclosure, and a combination partner (e.g., an antibody or another drug as explained below, also referred to as “therapeutic agent” or “co-agent”) may be administered independently at the same time or separately within time intervals. In some circumstances the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

In some embodiments, the subject in need thereof has or is believed to have a malignancy. Many types of malignancies can develop resistance mechanisms to evade attacks from endogenous NK cells, nonlimiting examples are provided herein. In some embodiments, the malignancy may include Acute Lymphoblastic Leukemia (ALL), AML, Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Carcinoid Tumor, Carcinoma, Cardiac Tumors, Atypical Teratoid/Rhabdoid Tumor, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer, Ependymoma, Esophageal Cancer, Esthesioneuroblastoma, Ewing Sarcoma, Extracranial Germ Cell Tumor, Extragonadal Germ Cell Tumor, Intraocular Melanoma, Retinoblastoma, Fallopian Tube Cancer, Fibrous Histiocytoma of Bone, Osteosarcoma, Gallbladder Cancer, Gastric Cancer, Gastrointestinal Carcinoid Tumor, Gastrointestinal Stromal Tumors (GIST) (Soft Tissue Sarcoma), Germ Cell Tumors, Central Nervous System Germ Cell Tumors, Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Hepatocellular Cancer, Histiocytosis (Langerhans Cell), Hodgkin Lymphoma, Hypopharyngeal Cancer, Islet Cell Tumors, Pancreatic Neuroendocrine Tumors, Kaposi Sarcoma (Soft Tissue Sarcoma), Renal Cell Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Merkel Cell Carcinoma, Mesothelioma, Metastatic Cancer, Metastatic Squamous Neck Cancer with Occult Primary, Midline Tract Carcinoma With NUT Gene Changes, Oropharyngeal Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myelogenous Leukemia (CML), Myeloid Leukemia, AML, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Recurrent Cancer, Rhabdomyosarcoma, Salivary Gland Cancer, Vascular Tumors, Small Intestine Cancer, Soft Tissue Sarcoma, T-Cell Lymphoma, Thymoma and Thymic Carcinoma, Transitional Cell Cancer of the Renal Pelvis and Ureter, Vaginal Cancer, Vulvar Cancer, or Wilms Tumor. In specific embodiments, the cancer is AML.

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the methods of the present disclosure and practice the claimed methods. The following working examples therefore, specifically point out embodiments of the present disclosure, and are not to be construed as limiting in any way the remainder of the disclosure.

### EXAMPLES

### Example 1

In a general sense, this invention provides an approach to modify therapeutic immune cells (for example NK cells, T and B lymphocytes, macrophages, etc.) to increase their resistance to drugs used to treat cancer and other diseases. Immune cells can be autologous cells, allogeneic cells from healthy donors or derived from pluripotent/multipotent stem cells (human embryonic stem cells (hESCs), induced pluripotent stem cells (iPSC), hematopoietic stem cells (peripheral blood, bone marrow or cord-blood isolated), etc.

NK cells have been derived from human iPSCs that were made resistant to the BCL2 inhibitor venetoclax. Venetoclax is a clinical therapy that is approved for treatment of certain cases of AML and CLL. Venetoclax and related BCL2 inhibitors (such as navitoclax) are in clinical trials with evidence of efficacy against other diverse hematological malignancies and solid tumors. While this therapy is beneficial, the tumors can develop resistance which leads to disease relapse. Also, the patient's normal blood cells can also be inhibited by the drug treatment, leading to anemia, immune deficiency, etc. This inhibition of normal cell survival also makes it impossible to administer cell-based therapies while patients are taking venetoclax. Here, the therapeutic immune cells were engineered to be resistant to venetoclax which allow more aggressive treatment of the malignancies by venetoclax without inhibition of cell product activity and with less risk of disease relapse.

The cells were genetically modified by one of the following strategies: 1) By inserting mutations in the DNA of genes or their corresponding RNA transcripts that encode for the proteins that are target of the drugs. This mutation can either block the binding of the drug thus limiting its toxic effect or increase the binding of specific molecules that are meant to enhance the effector functions of immune cells. 2) By depleting (knock out (KO)) the gene of interest when it is target of the drug or when it is part of the pathway targeted by the drug. The KO blocked the function of the target protein limiting the effect of the drug on immune cells. 3) By inserting a mutation in a specific gene blocking the binding of a drug or compound thus improving the survival of the mutated cells to the drug.

In this study, a point mutation was inserted on the gene Bcl-2 using undifferentiated human iPSCs that are subsequently differentiated into NK cells. Based on studies from drug resistant hematological tumors (1), the Glycine (G) in position 101 was changed into a Valine (V) that reduces the binding efficiency of venetoclax to the protein BCL2. BCL2 is a protein that blocks the activation of the apoptotic pathway and leads to prolonged cell survival. Once venetoclax binds to BCL2, it blocks the protein activity, such that BCL2 can no long can inhibit the apoptotic pathway. By inserting this mutation (the mutation can be either heterozygous or homozygous), the sensitivity of NK cells (or any other modified immune cells) to venetoclax, or related BCL2 inhibitors such as (but not limited to) navitoclax and obatoclax was reduced. This resistance to BCL2 inhibitors enables more effective combinatorial therapy using venetoclax plus iPSC-derived NK cells (or any other modified immune cells) for the improved treatment of hematological malignancies such as AML, CLL, and multiple myeloma. Improved treatment of solid tumors is also feasible.

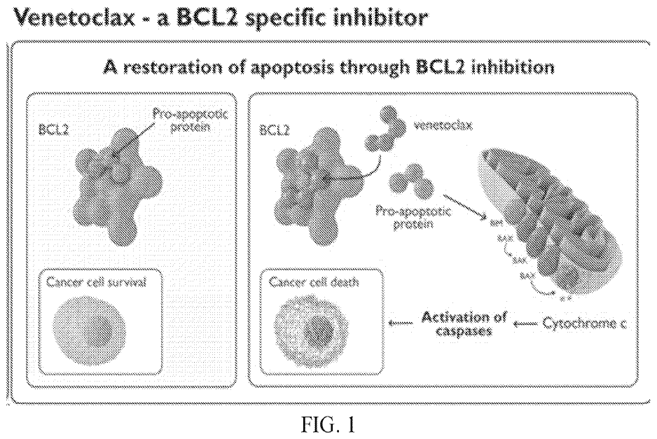

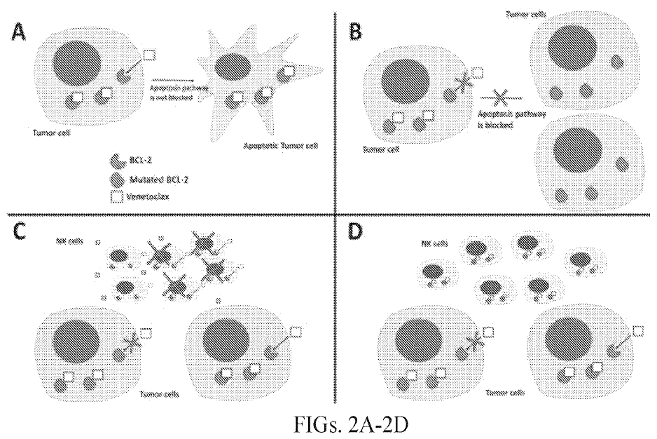

The normal activity of BCL2 is to prevent apoptosis (cell death) (FIG. 1). Increased activity of BCL2 leads to tumor development, as those cells fail to die, and accumulate in the body leading to inhibition of normal cell and tissue activities. Venetoclax is an inhibitor of BCL2 activity and can be used to treat malignancies such as CLL, AML, and multiple myeloma by counter-acting this process. However, the cancer cells can develop mutations that lead to resistance of venetoclax activity, leading to relapsed disease. Here, venetoclax resistant NK cells were generated that can be used to more effectively treat cancer cells by giving the engineered NK cells and venetoclax at the same time to prevent the development of venetoclax-resistant tumors (FIG. 2).

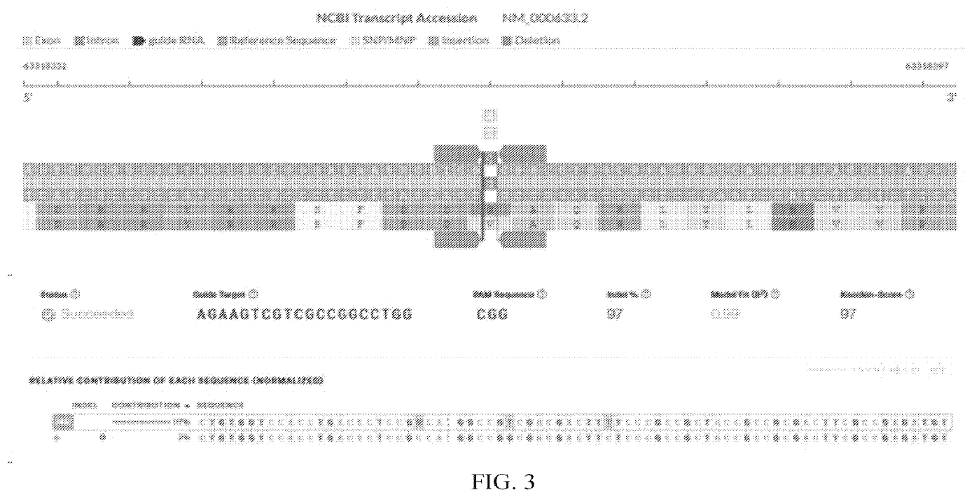

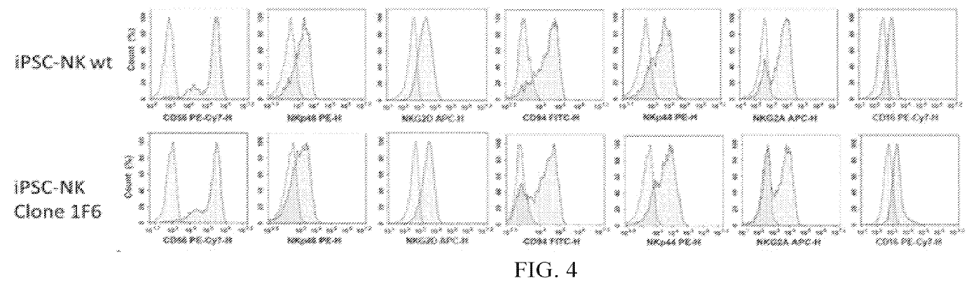

A genetic knock in (KI) was performed in iPSCs using CRISPR/Cas9 introducing the G101V mutation in the genome (FIG. 3, top panel). After single clone selection, the KI of a valine in place of a glycine at position 101 was confirmed by sequencing (FIG. 3, bottom panel). A G101V mutated iPSC clone (1F6) was differentiated into iPSC-derived NK cells. The clone selected showed a normal NK cell phenotype compared to the control wild type (WT cells) (FIG. 4).

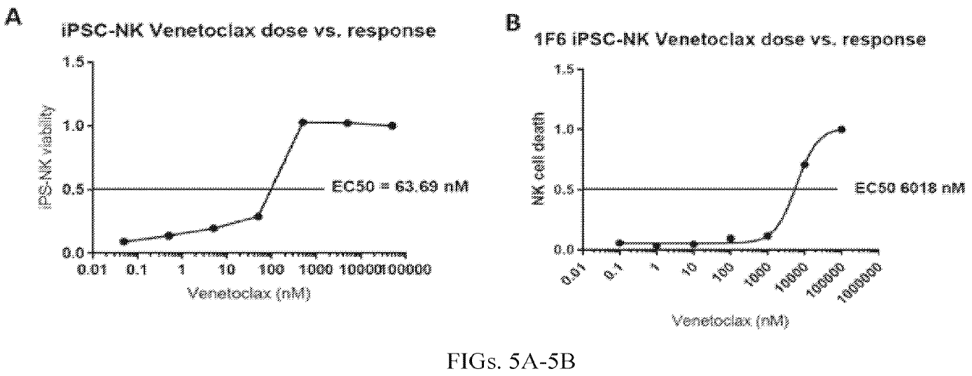

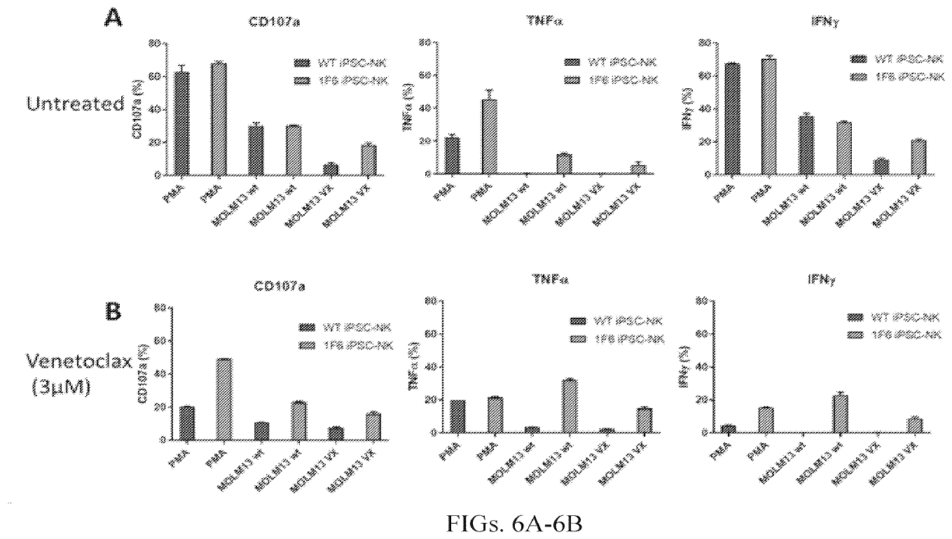

Successfully differentiation of NK cells from the mutated iPSC clone allowed us to analyze the resistance to venetoclax (FIG. 5) and compare the killing activity of the G101V iPSC-derived NK cells (also termed 1F6 NK cells) versus the WT cells (FIGS. 5 and 6A-6B). 1F6 NK cells demonstrated a thousand-fold improved resistance to venetoclax (FIG. 4) compared to wild-type (WT) iPSC-derived NK cells. Specifically, the effective dose-50 (EC50) where 50% of the NK cells are killed by venetoclax is 63.69 nM for the WT iPSC-derived NK cells and 6018 nM for the 1F6 NK cells with the engineered G101V mutation.

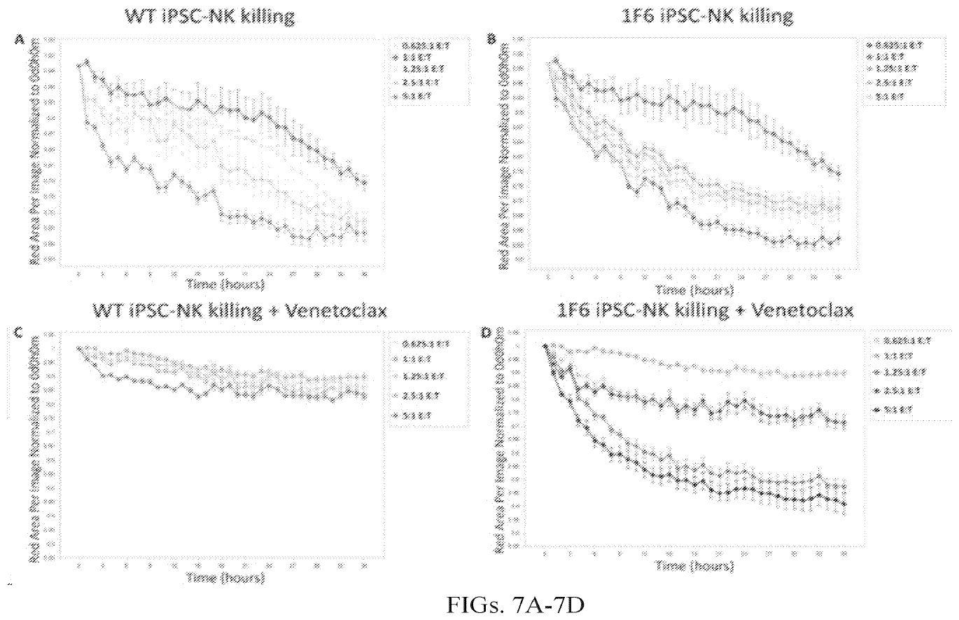

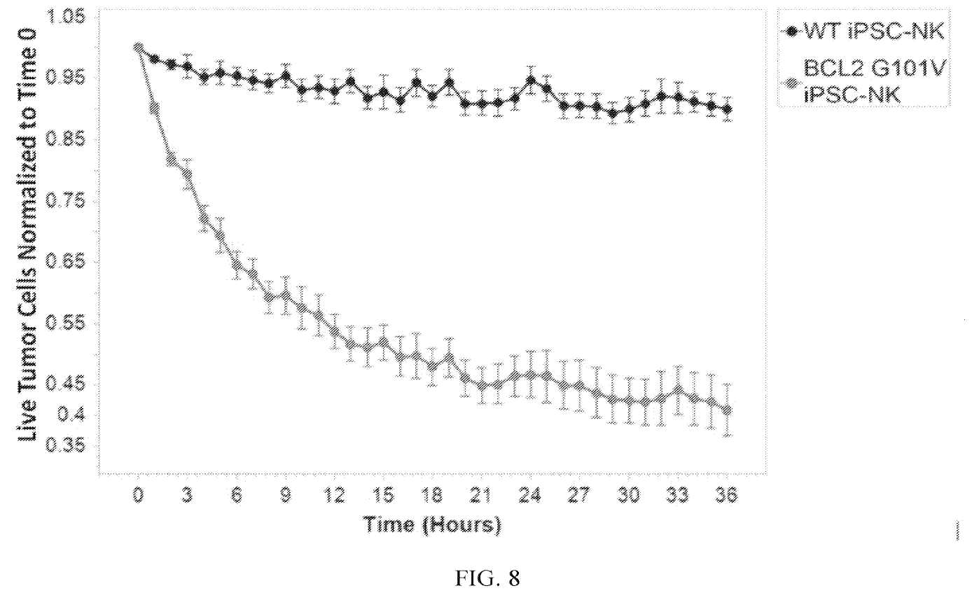

Next, a Molm13 AML cell line made resistant to venetoclax was used to perform activation and killing assays on 1F6 NK cells and WT iPSC-derived NK cells. 1F6 NK cells showed improved degranulation and secretion of more TNFα and IFNγ compared to WT NK cells (FIGS. 6A-6B). Subsequent studies did a longer-term cytotoxicity test using 1F6 NK cells and WT NK cells in a coculture with Molm13 VX or WT in the presence of venetoclax. 1F6 NK cells performed better that WT-NK cells (FIG. 7) demonstrating that the mutation G101V can improve the killing efficiency of NK cells when exposed to venetoclax.

Venetoclax treatment and/or other Bcl2 inhibitors such as navitoclax is now approved or in clinical trials for hematological malignancies including AML, CLL, ALL, multiple myeloma, and lymphoma. These agents are also in clinical trials for treatment of diverse solid tumors including (but not limited to) breast cancer, lung cancer, bladder cancer, liver cancer, colorectal cancer, and others. NK cell-based therapies are also being pursued for most of these malignancies. Therefore, we would envision a commercial application where iPSC (or other source of NK cell or another immune cell) would be engineered to be venetoclax resistant. These cells would then be administered while a patient is getting venetoclax treatment to improve the NK cell-mediated anti-tumor activity and prevent development of venetoclax-resistant disease.

The commercial application of this technology can be quite broad. As noted, there are several Bcl2 inhibitors either FDA approved or in trials. This approach would apply to any of these. More broadly, these studies demonstrate the concept and ability to make immune cells (in particular iPSC-derived cells) resistant to therapeutic drugs that are otherwise toxic to the administered cell product-so claims that cover general drug resistance would be appropriate. In addition to diverse tumor applications (as above), the cell products that can be engineered for drug resistance include T cells and macrophages, in addition to NK cells. The source of the engineered cell product can be peripheral blood-derived, cord blood-derived, bone marrow-derived, iPSC-derived or hESC-derived.

### Example 2

This example demonstrates that NK cells (e.g., iPSC-derived NK cells) can be engineered to generate venetoclax-resistance for use in combination with venetoclax therapy to improve treatment of AML.

To create venetoclax-resistant iPSC-derived NK cells, the BCL2 G101V mutation was introduced into iPSCs using CRISPR/Cas9 technology. Homozygous BCL2 G101V iPSC-derived NK cells (BCL2G101V/G101V) were used to demonstrate that venetoclax-resistant NK cells could be combined with venetoclax to improve NK cell-mediated killing of AML cells in vitro and in vivo.

### Methods

### Cell Lines and Cell Culture

Human iPSCs derived from umbilical cord blood CD34+ cells were derived and maintained as undifferentiated cells on Matrigel-coated tissue culture flasks in mTeSR media (STEMCELL™ Technologies), as previously described (D. L. Hermanson, et al., 2016; D. A. Knorr, et al., 2013; H. Zhu, et al., 2020; Y. Li, et al., 2018; H. Zhu, et al., 2020). K562 and MOLM13 cell lines were obtained from ATCC. K562 and MOLM13 were cultured in RPMI 1640 media (Thermo Fisher Scientific) supplemented with 2 mM glutamine (Sigma) and with 10% heat-inactivated fetal bovine serum (FBS) (Sigma).

### Generation of Venetoclax Resistant AML Cells

The MOLM13 cells lines were cultured continuously in increasing concentrations of venetoclax over 6 weeks to generate and select for venetoclax-resistant cells. The resulting cells could be maintained in 3 μM venetoclax without toxicity. The resistant cells were cultured continuously in the presence of venetoclax.

BCL2 G101V Knock-In in iPSCs

Introduction of the BCL2 G101V mutation in iPSCs was completed using CRISPR/Cas9 technology. A gRNA targeting the DNA encoding codon 101 the BCL2 gene was designed. crRNA-1 #: CAAGGGCTGCATGACTGGCT, (SEQ ID NO:1) the DNA templates for the BCL2 G101V mutation was co-transfected into the iPSCs using a Nucleofector 2b (Lonza, AAB-1001) with Human Stem Cell Nucleofector Kit (Lonza, VPH-5012). 24 hours after transfection, single cells were isolated and seeded to a 96-well plate at concentration of 2.5 cells/ml (200 μl plated per well) for single clone selection. Mutations in the BCL2 gene were identified by Sanger Sequencing and PCR with restriction digest using the restriction enzymes SalI (cuts G101V mutant DNA sequence) and NaeI (cuts WT DNA sequence). Clones homozygous and heterozygous for the BCL2 G101V mutation were identified by the ICE CRISPR analysis tool (Synthego).

Derivation and Expansion of NK Cells from iPSCs

The derivation of NK cells from iPSCs has been previously described (D. A. Knorr, et al., 2013; H. Zhu, et al., 2019). Briefly, 8,000 TrypLE-adapted iPSCs were seeded in 96-well round-bottom plates with APEL2 media (STEMCELL™ Technologies, Vancouver, BC, Canada) containing 40 ng/ml human Stem Cell Factor (SCF), 20 ng/ml human Vascular Endothelial Growth Factor (VEGF), and 20 ng/ml recombinant human Bone Morphogenetic Protein 4 (BMP-4). After day 8 of hematopoietic differentiation, spin embryoid bodies were then directly transferred into each well of uncoated 24-well plates under a condition of NK cell culture. Cells were then further differentiated into NK cells using 5 ng/mL IL-3 (first week only), 10 ng/ml IL-15, 20 ng/ml IL-7, 20 ng/mL SCF, and 10 ng/mL flt3 ligand for 28-32 days. iPSC-derived NK cells were expanded using irradiated K562-IL21-41BBL cells, also termed artificial antigen presenting cells (aAPCs) (C. J. Denman, et al., 2012).

### Flow Cytometry

Flow cytometry was done on a BD FACS Calibur, BD LSRII or NovoCyte 3000, and data were analyzed using FlowJo or NovoExpress.

### Antibodies

The following antibodies were used for flow cytometry (all anti-human): CD16-PE (BD Biosciences, 560995, clone 3G8), NKG2D-APC (BD Biosciences, 558071, clone 1D11), NKp44-PE (BD Biosciences, 558563, clone p44-8), NKp46-PE (BD Biosciences, 557991, clone 9E2), TRAIL-PE (BD Biosciences, 565499, clone YM366), FAS ligand-PE (BD Biosciences, 56426, clone NOK-1), NKG2A-PE (Beckman Coulter, IM3291U, clone Z199), CD158a,h (KIR2DL1, KIR2DS1)-PE (Beckman Coulter, A09778, clone EB6B), Interferon gamma-Pacific Blue (BioLegend, 502522, clone 4S.B3), CD107a-APC (BioLegend, 328620, clone H4A3).

### CD107a/IFN-Gamma NK Cell Activation Assay:

NK cells were incubated with or without cancer targets (MOLM13 cells) at 1:2 effector to target (E:T) ratios. CD107a-APC antibody was added to each well and allowed to incubate for 1 hour, followed by adding GolgiStop and GolgiPlug for additional 2 hours incubation. At the completion of incubation, cells were washed with FACS buffer, were stained with CD56-PE and live/dead Aqua staining. Cells were then fixed with fixation buffer for 10 minutes on ice, following by permeabilization with perm/wash buffer for 10 minutes at 4° C. Cells were washed and stained with interferon-gamma (IFN-gamma)-Pacific Blue for 30 min at 4° C., then finally washed prior to analysis by flow cytometry.

### Cytotoxicity Assays:

CellEvent™ Caspase-3/7 Green Flow Cytometry assay: Target cells were pre-stained with CellTrace™ Violet (Thermo-Fisher Scientific, C34557) at a final concentration of 5 μM in PBS for 15 min at 37° C. After staining, the cells were washed in complete culture medium prior to being mixed with NK cell cultures at the indicated effector to target (E:T) ratios. After a brief centrifugation, co-cultures were incubated at 37° C. for 3.5 hrs. Afterwards, CellEvent™ Caspase-3/7 Green Detection Reagent (Thermal Fisher Scientific, C10423) was added for an additional 30 min of culture for a total incubation time of 4 hours. During the final 5 minutes of staining, SYTOX™ AADvanced™ dead cell stain solution (Thermal Fisher Scientific, S10349) was added and mixed gently. Cells were then analyzed by flow cytometry.

IncuCyte® Caspase-3/7 Green Apoptosis assay: Target cells were labeled with CellTrace™ Far Red (ThermoFisher, C34564). Adherent target cells were seeded in a 96-well plate at a density of 4000 cells/well 24 hr before addition of IncuCyte® Caspase-3/7 Green Apoptosis Assay Reagent (Essen Bioscience, 4440) to each well diluted by a factor of 1,000. Non-adherent target cells were seeded in fibronectin coated 96-well plates at a density of 30,000 to 50,000 cells/well and further incubated at room temperature for 30 min before the addition of IncuCyte® Caspase-3/7 Green Apoptosis Assay Reagent. After incubation, NK cells were added at various E:T ratios and monitored on the IncuCyte® ZOOM to acquire images every 1 h for adherent cells and every 30 min for non-adherent cells. Experiments were performed with 3 independent biological triplicates. The cytotoxicity of target cells was analyzed by quantifying red cell number and/or overlay of Caspase 3/7 (green) within the red cells.

### RNA Isolation and Quantitative Real-Time PCR

Total RNA was isolated from cells using the RNeasy mini-Kit (Qiagen, 74104) according to the manufacturer's protocol. RNA quantification and qualification were performed using Nanodrop for checking RNA purity (OD260/OD280). For qRT-PCR complementary DNA (cDNA) was reverse transcribed from RNA isolated as above using the iScript gDNA clear cDNA Synthesis Kit (Bio-Rad, 1725034) according to the manufacturer's instructions. cDNA was used as a template in qPCR experiments using specific primers (500 nM) and Sso Advanced Universal SYBR Green Super Mix as the detection chemistry (Bio-Rad, 1725270). The thermal profile included one phase at 95° C. for 3 min, second phase of 40 cycles at 95° C. for 15 s, 60° C. for 30 s. Melting curve analysis was run after every experiment. The experiments were carried out and analyzed with Bio-Rad CFX 96 and software (Bio-Rad). Cycle threshold (Ct) values were determined, and relative mRNA contents were inferred from normalization of the gene of interest expression to that of the housekeeping gene GAPDH (ΔCt). Relative expression results were plotted as (2-ΔCt). Forward and reverse primer sequences are:

GAPDH-F:
(SEQ ID NO: 2)
GTCTCCTCTGACTTCAACAGCG,
GAPDH-R:
(SEQ ID NO: 3)
ACCACCCTGTTGCTGTAGCCAA.
BCL2-F:
(SEQ ID NO: 4)
CCACTTACCTGAATGACCACC,
Alt. BCL2-F:
(SEQ ID NO: 5)
GTGGCCTTCTTTGAGTTCGG,
BCL2-R:
(SEQ ID NO: 6)
GCCGTACAGTTCCACAAAGG,
BCL-XL-F:
(SEQ ID NO: 10)
CCACTTACCTGAATGACCACC;
BCL-XL-R:
(SEQ ID NO: 7)
TTCCGACTGAAGAGTGAGCC,
MCL1-F:
(SEQ ID NO: 8)
CCAAGAAAGCTGCATCGAAC,
MCL1-R:
(SEQ ID NO: 9)
AACTCCACAAACCCATCCC.

### Mouse Xenograft Study

Eight-week-old NOD/SCID/γc−/− (NSG) mice (Jackson Laboratories, n=5 per group) were used for in vivo experiments. Mice received 2.5×105 luciferase-expressing MOLM13 tumor cells intravenously. Mice were sub-lethally irradiated (225 cGy) 4 days after tumor engraftment. 107 NK cells/mouse were injected IV on the day of irradiation. NK cells were supported by the injection of IL-2 and IL-15 as reported previously 27, 28. Mice were treated with venetoclax (50 mg/kg) or vehicle daily for 21 days. Tumor burden was determined by BLI using the Xenogen IVIS Imaging system. Mice were sacrificed when loss of ability to ambulate was observed. All mice were housed, treated, and handled in accordance with the guidelines set forth by the University of California, San Diego Institutional Animal Care and Use Committee and the National Institutes of Health's Guide for the Care and Use of Laboratory Animals.

### Statistics

Unpaired student's t-test and ANOVA were used to quantify statistical deviation between experimental groups. The log-rank test was to analyze the Kaplan-Meyer survival analyses in the in vivo study. Asterisks denote significant differences *P<0.05; **P<0.01; ***P<0.001 for comparisons as indicated in figure legends.

### Results

Homozygous CRISPR-Cas9 mediated knock-in of the BCL2 G101V mutation in iPSCs and differentiation of BCL2G101V/G101V iPSCs-NK cells.

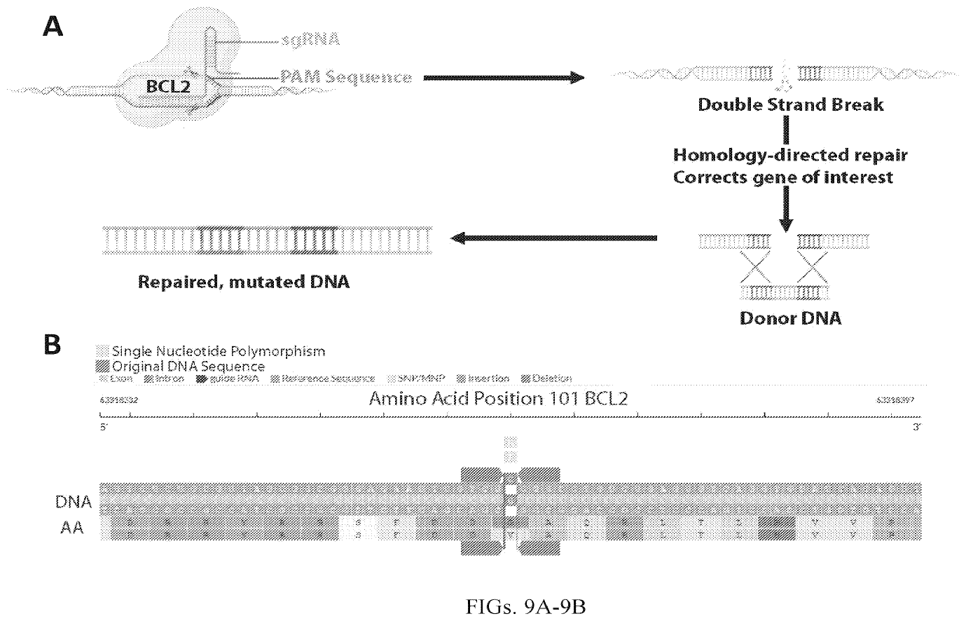

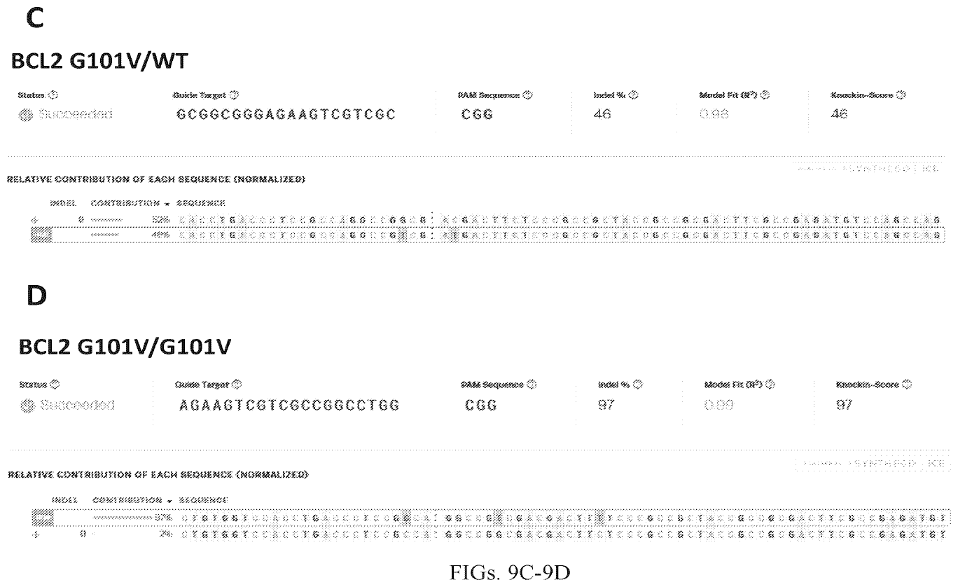

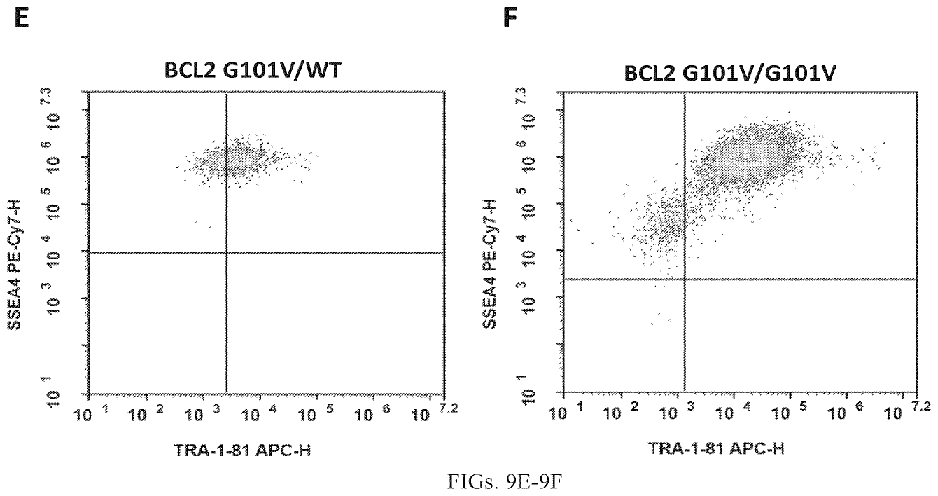

To generate iPSC-derived NK cells with an improved ability to tolerate exposure to venetoclax, CRISPR/Cas9 technology was first employed to knock-in the BCL2 G101V mutation into undifferentiated iPSCs (FIG. 9A). CRISPR/Cas9 editing to mediate homology directed repair was used to introduce the BCL2 G101V mutation into both BCL2 alleles of iPSCs by electroporating Cas9, a guide RNA targeting the BCL2 gene, and a donor plasmid carrying the DNA sequencing for the BCL2 G101V mutation (FIG. 9B). iPSCs heterozygous and homozygous for the BCL2 G101V mutation were then detected by screening PCR and restriction digest utilizing a restriction fragment length polymorphism generated by the mutation. The heterozygous or homozygous BCL2 G101V mutation was then confirmed by Sanger sequencing (FIGS. 9C-9D). The selected single cell iPSC clones were then expanded. The expanded clones were then tested for pluripotency maker expression and were shown to be SSEA4 and TRA-1-81 positive, demonstrating that they retained pluripotency following this manipulation (FIGS. 9E-9F). The BCL2G101V/G101V iPSCs were then differentiated to NK cells (FIG. 10).

WT iPSC-derived NK cells and BCL2 G101V/G101V iPSC-derived NK cells demonstrate typical NK cell surface receptor phenotypes and function.

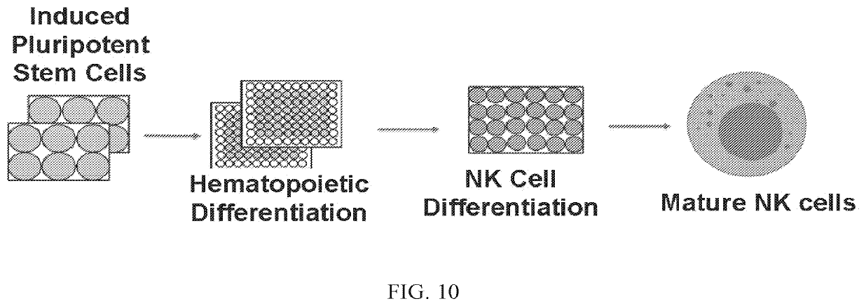

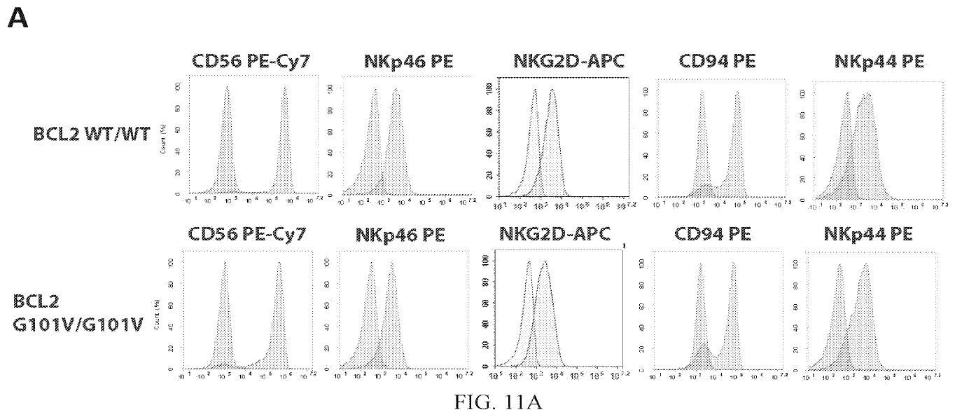

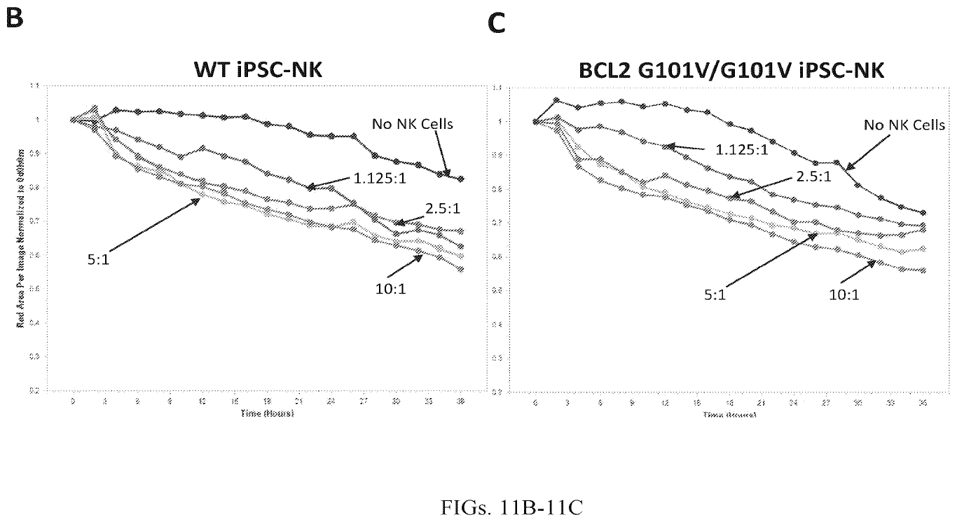

WT iPSC-derived NK cells and BCL2 G101V/G101V iPSC-derived NK cells were analyzed by flow cytometry for standard NK cell surface proteins. This phenotyping demonstrated that both the WT and engineered iPSC-derived NK cells had a typical NK cell phenotype with no differences in expression of the receptors analyzed (FIG. 11A). Functional analyses were also completed with both groups of NK cells. There was no difference seen in cytotoxicity against K562 tumor cells between BCL2 G101V/G101V iPSC-derived NK cells and WT iPSC-derived NK cells (FIGS. 11B-11C).

BCL2 G101V iPSC-derived NK cells are resistant to venetoclax and maintain function when treated with venetoclax.

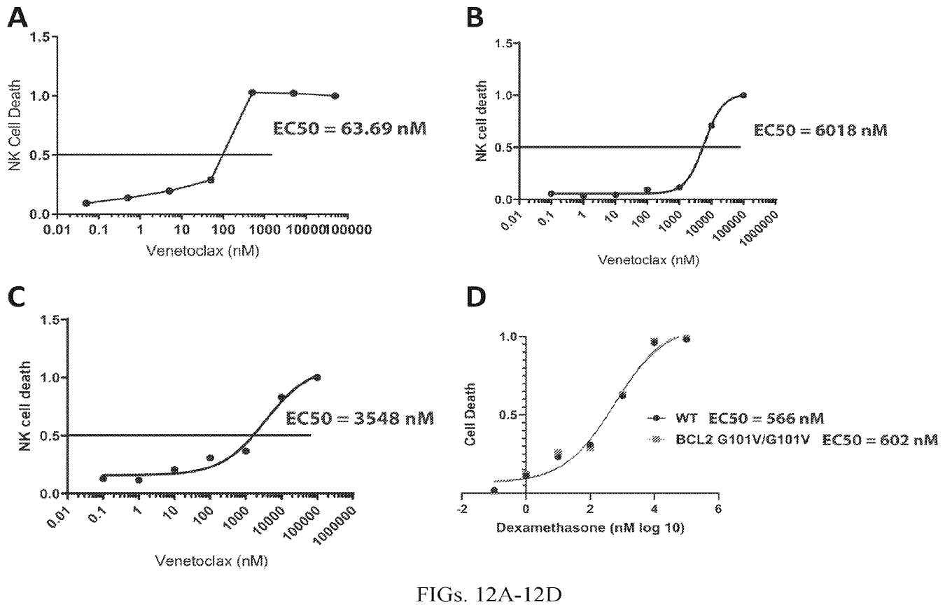

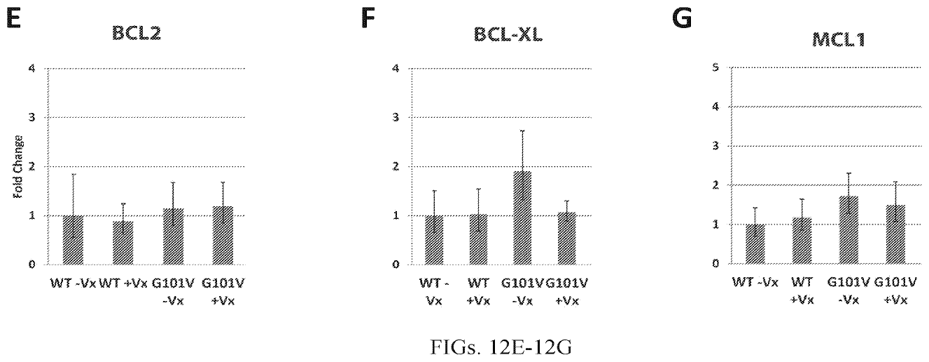

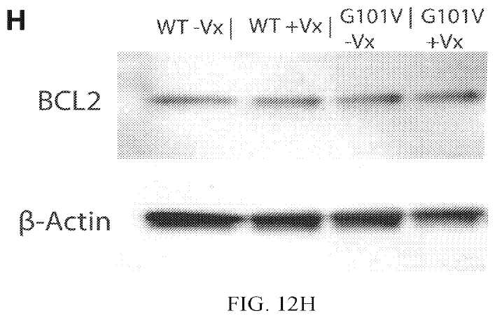

Resistance to venetoclax of the heterozygous and homozygous BCL2 G101V iPSC-derived NK cells was tested in cell survival assays. The homozygous BCL2G101V/G101V NK cells had an EC50 of 6018 nM to venetoclax compared to 3548 nM and 63.69 nM for the heterozygous BCL2G101V/WT NK cells and WT BCL2WT/WT NK cells, respectively (FIGS. 12A-12C). Additionally, it was shown that the resistance to venetoclax generated by the BCL2 G101V mutation does not confer resistance to other cytotoxic agents, such as dexamethasone. For example, treatment with other cytotoxic agents including dexamethasone showed IC50s that were not significantly different between the BCL2WT/WT NK cells and BCL2G101V/G101V NK cells (FIG. 12D).

To determine whether the BCL2 G101V point mutation affects expression levels of the BCL2 gene or protein, qualitative real-time PCR and immunoblot analyses were performed. The results demonstrated that BCL2, MCL1 and BCL-XL mRNA and protein levels did not differ between BCL2WT/WT NK cells and BCL2G101V/G101V NK cells in the presence or absence of venetoclax (FIGS. 12D-12H).

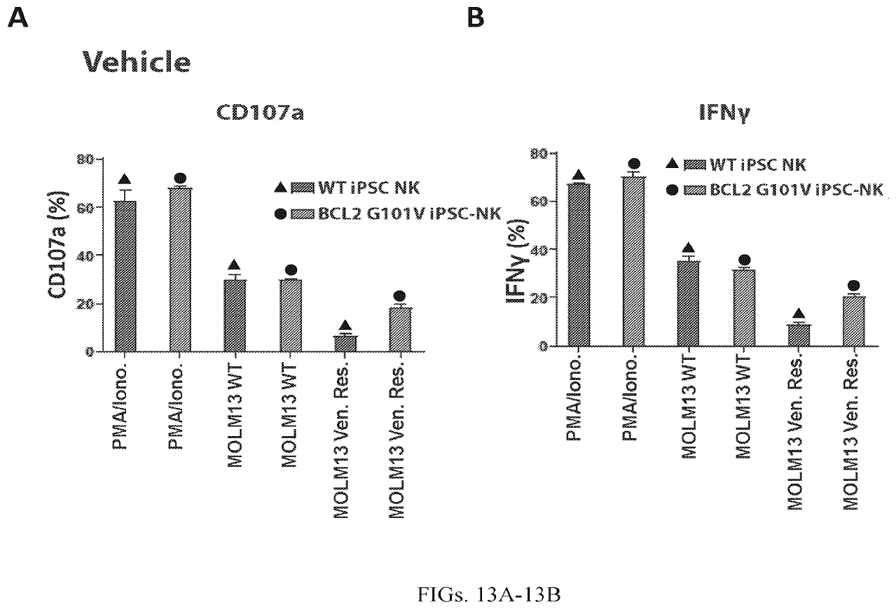

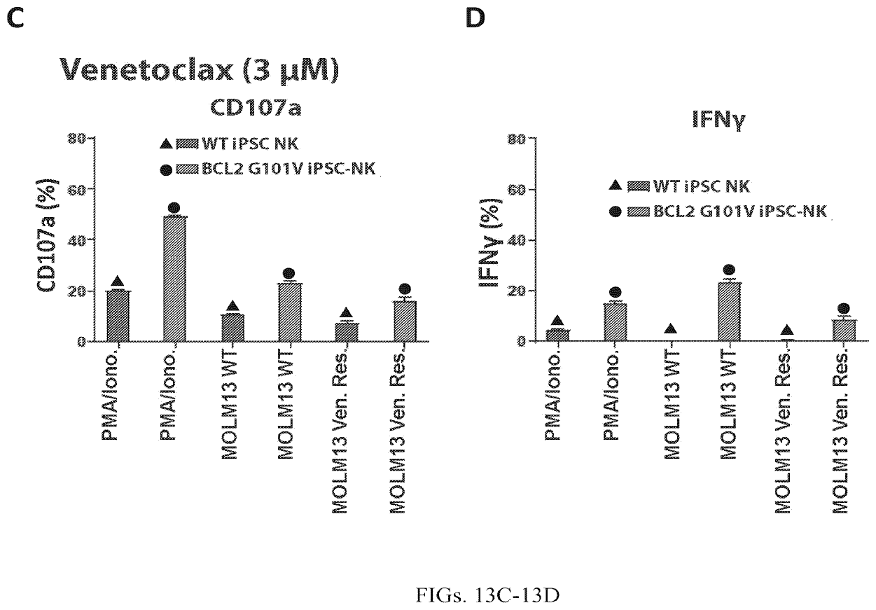

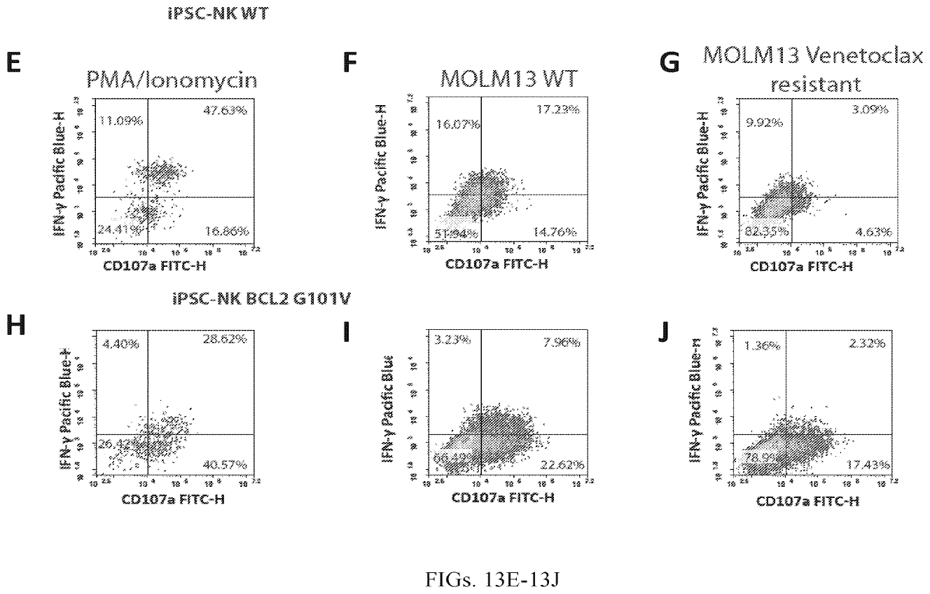

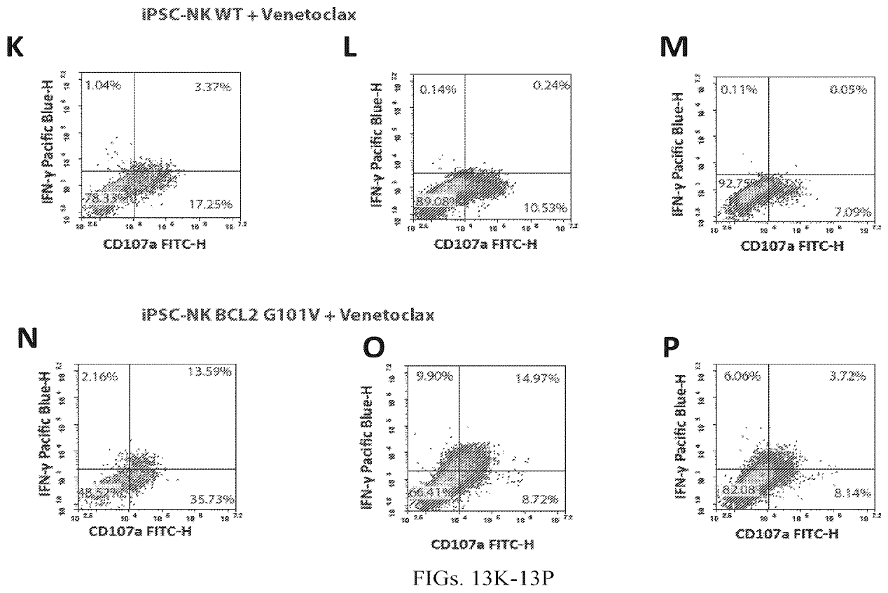

Additional functional analyses demonstrated that activity of the BCL2G101V/G101V iPSC-derived NK cells was preserved upon exposure to venetoclax. CD107a cell surface expression (an indicator of NK cell degranulation), and IFN-gamma production by WT iPSC-derived NK cells stimulated by MOLM13 AML tumors cells was reduced nearly 3-fold upon addition of venetoclax. In contrast, CD107a expression on BCL2G101V/G101V iPSC-derived NK cells and their IFN-gamma production was not significantly affected by addition of venetoclax (FIGS. 13A-13P).

BCL2 G101V iPSC-derived NK cells are resistant to inhibition by venetoclax and maintain normal cytotoxicity during venetoclax treatment.

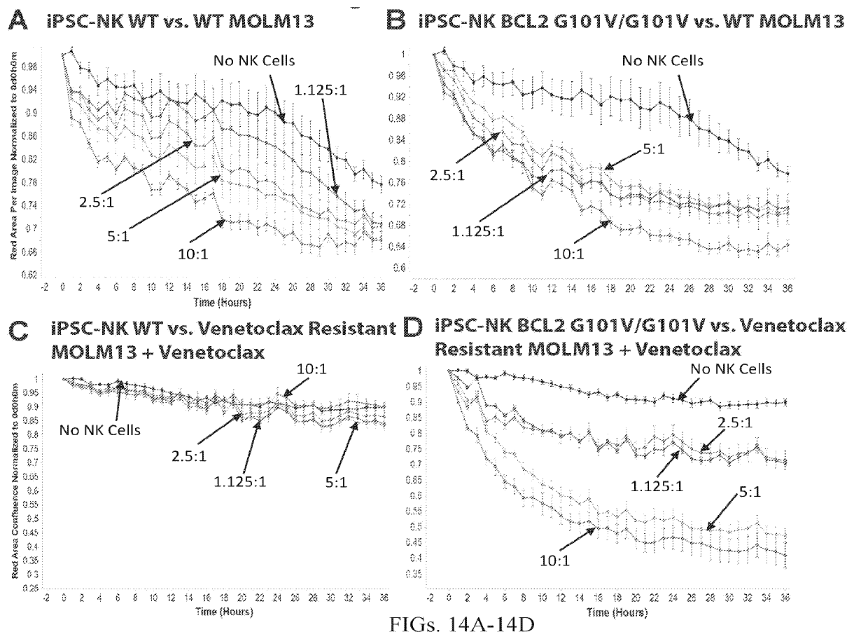

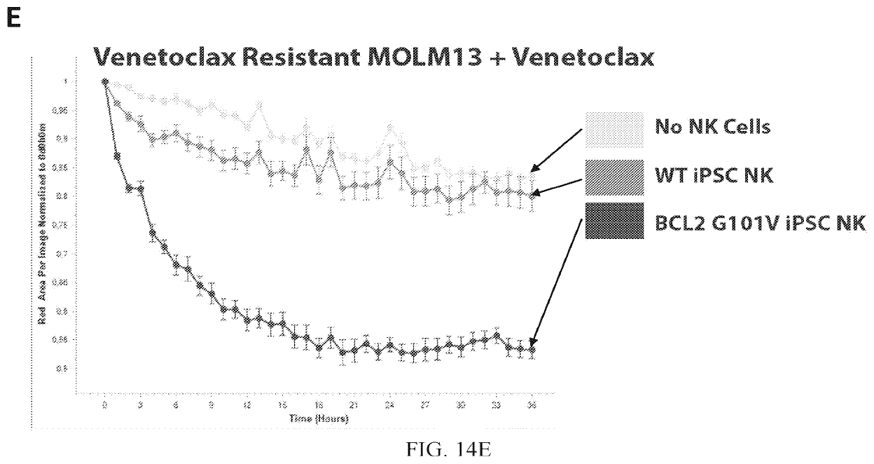

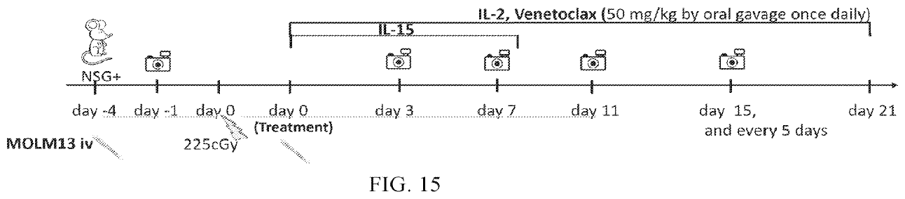

For longer-term assays to examine the ability of BCL2G101V/G101V iPSC-derived NK cells to mediate effective killing of venetoclax-resistant AML cells, MOLM13 cells that are venetoclax-resistant were generated. Specifically, these venetoclax-resistant MOLM13 demonstrate over 100-fold increased resistance so they survive at 3 μM venetoclax compared to the WT MOLM13 cells with an IC50 of 24 nM. The BCL2G101V/G101ViPSC-derived NK cells' resistance to venetoclax was also demonstrated in longer term 36-hour cytotoxicity assays using the Incucyte imaging system. Initial studies that compared BCL2G101V/G101ViPSC-derived NK cells and BCL2WT/WT iPSC-derived NK cells found similar activity against WT-MOLM13 cells when no venetoclax was present (FIGS. 14A-14B). However, when the WT or engineered iPSC-derived NK cells were treated with 1.5 μM venetoclax or vehicle for 24 hours prior to co-incubation and during the 36-hour assay, only the BCL2G101V/G101V iPSC-derived NK cells mediated effective anti-AML activity, whereas the WT iPSC-derived NK cells were inhibited by the drug treatment (FIGS. 14C-14D). A direct comparison between the WT and engineered iPSC-derived NK cells further demonstrates the improved activity of the BCL2G101V iPSC-derived NK cells against the venetoclax-resistant MOLM13 cells during venetoclax treatment (FIG. 14E).

BCL2 G101V iPSC-derived NK cells mediate improved tumor killing in combination with venetoclax in vivo.

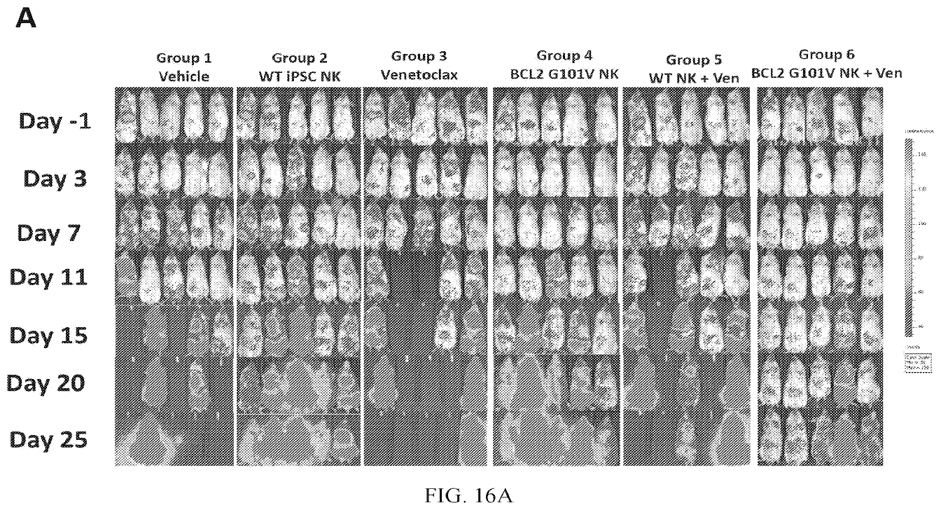

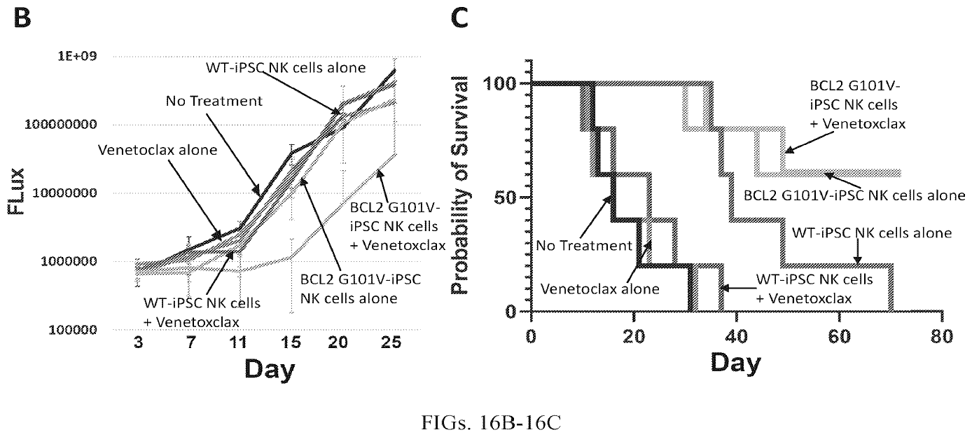

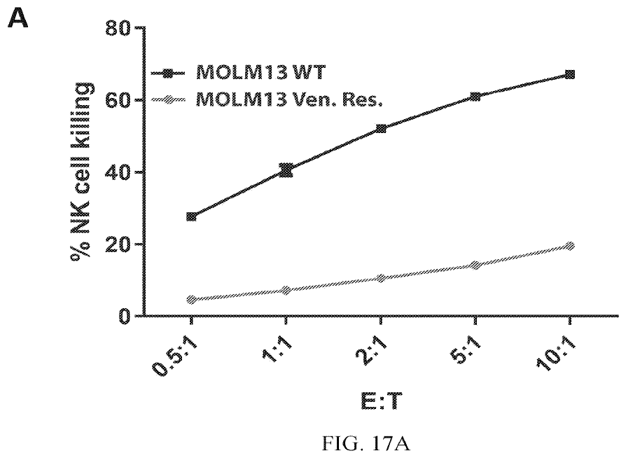

To evaluate the anti-tumor activity of BCL2G101V/G101V NK cells in vivo, BCL2G101V/G101V iPSC-derived NK cell killing of MOLM13 AML cells was assessed in a mouse xenograft tumor model. Mice were injected intravenously (IV) with luciferase-expressing, venetoclax-resistant MOLM-13 cells and then four days later day received a single IV injection of 107 WT or BCL2G101V/G101V iPSC-derived NK cells. As in previous studies, IL-15 was dosed daily for 7 days, and IL-2 was dosed every third day for 21 days to promote in vivo NK cell survival (FIG. 15). Venetoclax (50 mg/kg) or vehicle was dosed daily by oral gavage for 21 days (FIG. 15). Tumor growth was monitored by bioluminescent imaging (BLI) (FIG. 16A). In mice not treated with venetoclax, administration of WT iPSC-derived NK cells alone compared to no treatment significantly improved survival, but this effect was lost with the addition of venetoclax to the WT iPSC-derived NK cells. However, the BCL2G101V/G101V NK cell treated mice had prolonged survival both with and without venetoclax treatment (FIG. 16C), demonstrating effective anti-tumor activity of the BCL2G101V/G101ViPSC-derived NK cells even when venetoclax treatment was done during NK cell administration.

Venetoclax-resistant AML cell develops cross-resistance to WT-NK cell-mediated killing even without venetoclax exposure.

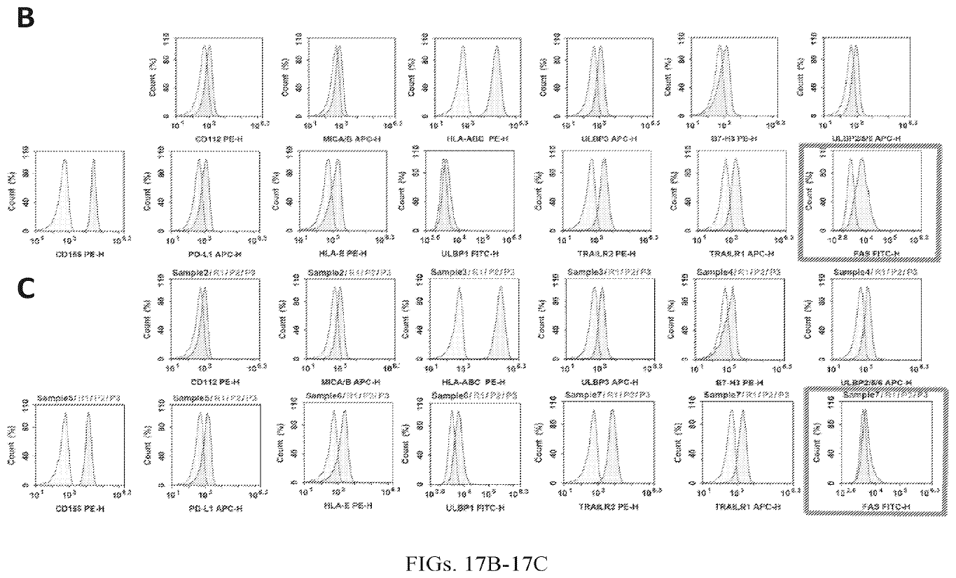

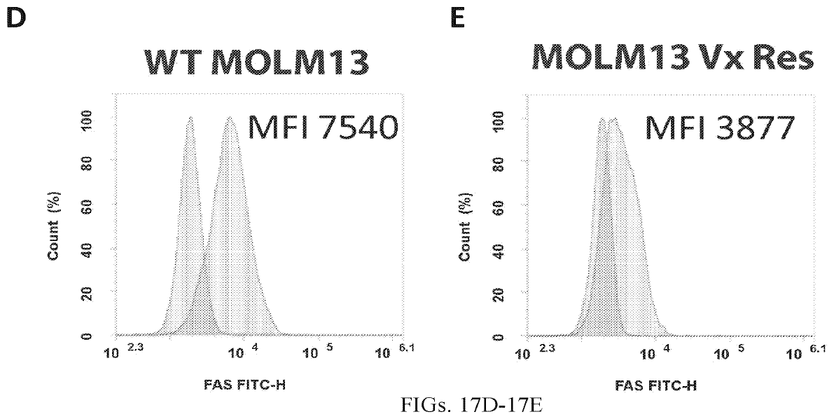

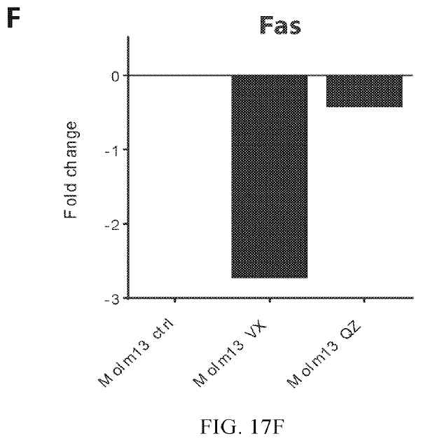

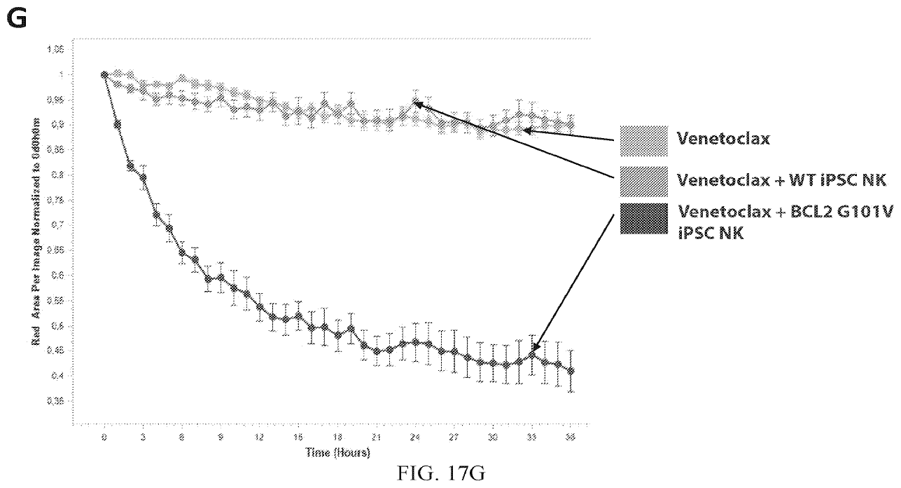

Based on the observations that BCL2G101V/G101V iPSC-derived NK cells had improved cytotoxicity against venetoclax-resistant AML cells and that the combination of venetoclax with BCL2G101V/G101V iPSC-derived NK cells had better long-term tumor control in vivo compared to BCL2G101V/G101V iPSC-derived NK cells alone, changes in AML cells susceptibility to NK cell killing as they develop resistance to venetoclax were investigated (FIGS. 14E and 16C). Interestingly, we found the venetoclax-resistant MOLM13 cells were more resistant to NK cell-mediated killing even without venetoclax treatment (FIG. 17A). NK cell activating and inhibitory ligands and death receptors on the surface of MOLM13 cells were examined by flow cytometry (FIGS. 17B-17C). While expression of most of these antigens were the same in the WT and venetoclax-resistant MOLM13 cells, a significant decrease in Fas was found on the venetoclax-resistant cells, both by flow cytometry and RT-PCR analyses (FIGS. 17B-17F). MOLM13 cells made resistant to quizartinib did not show the same down regulation of Fas expression (FIG. 17F). Notably, the venetoclax-resistant MOLM13 cells were resistant to killing by venetoclax alone and the combination of venetoclax and WT iPSC-derived NK cells. However, the combination of venetoclax and BCL2G101V/G101V iPSC-derived NK cells were able to effectively kill the venetoclax resistant MOLM13 cells (FIG. 17G).

### Discussion

Venetoclax has become an important tool in our armamentarium against AML and other malignancies. However, resistance to this treatment is unfortunately relatively common. Multiple clinical trials also demonstrate that adoptive transfer of allogeneic NK cells can have potent anti-AMI, activity. Therefore, the combination of venetoclax plus allogeneic NK cells could provide a potent new strategy from treatment of AML. However, inhibition of NK cell survival and cytotoxic function by venetoclax must be avoided to allow for the therapies to function synergistically. Venetoclax treatment leads to lymphopenia as BCL-2 is required for NK cell survival and venetoclax induces apoptosis of NK cells. Here this work demonstrates that engineering a specific mutation of the BCL2 gene in iPSC-derived NK cells that generates resistance to venetoclax effectively prevents toxicity to NK cells from venetoclax treatment. Specifically, a mutation found in chronic lymphocytic leukemia cells resistant to venetoclax (BCL2 G101V) was introduced and this mutation was validated to have resulted in venetoclax resistance in vitro and in vivo. As reported here and by others, exposure to venetoclax causes NK cell apoptosis and failure of NK cell tumor killing. A dose of 400 mg/day of venetoclax leads to a serum concentration of 2.3 μmol/L, which as shown here, was toxic to unengineered NK cells.

Safety is another key consideration in development of novel cell therapies that mutate or overexpress BCL2 to generate venetoclax resistance. BCL-2 overexpression in lymphocytes can lead to the development of lymphoma (T. Lackrai, et al., 2018; G. P. Linette, et al., 1995; M. Klanova, et al., 2020). Taking this into account, an important safety benefit of our CRISPR editing in the BCL2 G101V mutation approach is that BCL-2 is expressed at physiologic levels. Here, BCL2 was not overexpressed, rather the endogenous BCL-2 locus is mutated to generate venetoclax resistance. Compared to other approaches in which BCL-2 is overexpressed, the risk of transformation is therefore lower. There was no increased proliferation at baseline of the BCL2G101V/G101V iPSC-derived NK cells and no increased resistance to other cytotoxic therapies such as dexamethasone.

These results also demonstrate not only that venetoclax can potentiate NK cell killing of AML, but that cross resistance can develop, with venetoclax-resistant cells being more resistant to NK cell killing. These venetoclax resistant AML cells were found to down-regulate Fas (Faslo). The resistance to NK cell killing was overcome by the engineered mutation of BCL2 in the iPSC-derived NK cells, as the Faslo AML cells that are resistant to venetoclax were still able to be effectively killed by the combination of venetoclax and BCL2G101V/G101V iPSC-derived NK cells in vitro and in vivo.

In summary, this example demonstrate that a targeted mutation of BCL-2 prevents venetoclax mediated death of engineered iPSC-derived NK cell death and these drug-resistant NK cells have significantly improved anti-AML activity in the presence of ongoing venetoclax treatment. Additionally, this example demonstrates that AML cells that developed resistance to venetoclax became less sensitive to NK cell-mediated killing through down-regulation of Fas, but retained sensitivity to BCL2G101V/G101V iPSC-derived NK cells.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1. A method of treating or preventing a disease or disorder in a subject in need thereof, the method comprising administering to a subject in need thereof an effective amount of cytotoxic-resistant modified natural killer (NK) cells and a cytotoxic therapy.

2. The method of claim 1, wherein the cytotoxic therapy is venetoclax.

3. The method of claim 1, wherein the modified NK cells are BCL2 G101V mutated NK cells.

4. The method of claim 3, wherein the BCL2 G101V mutated cells are effective to mediate a resistance to the cytotoxic therapy.

5. The method of claim 1, wherein the disease or disorder is cancer.

6. The method of claim 5, wherein the cancer is acute myeloid leukemia (AML).

7. The method of claim 6, wherein the AML is resistant or refractory to venetoclax.

8. The method of claim 1, wherein administering the effective amount of modified NK cells and the cytotoxic therapy is effective to treat the disease or disorder without inhibiting cell product activity.

9. The method of claim 1, wherein the modified NK cells are derived from induced pluripotent stem cell (iPSC)-derived immune cells.

10. A purified cell composition comprising cytotoxic-resistant modified NK cells.

11. The purified cell composition of claim 10, wherein the modified NK cells are BCL2 G101V mutated NK cells.

12. The purified cell composition of claim 11, wherein the modified NK cells comprise homozygous BCL2 G101V mutations.

13. The purified cell composition of claim 11, wherein the modified NK cells comprise heterozygous BCL2 G101V mutations.

14. The purified cell composition of claim 10, wherein the modified NK cells are induced pluripotent stem cell (iPSC)-derived natural killer cells.

15. The purified cell composition of claim 10, wherein the modified NK cells comprise homozygous inactivating mutations in a cytokine-inducible SH2-containing protein (CISH) gene.

16. The purified cell composition of claim 10, wherein the modified NK cells comprise a chimeric antigen receptor.

17. A pharmaceutical composition comprising an effective amount of BCL2 G101V mutated NK cells and venetoclax.

18. The pharmaceutical composition of claim 17, wherein the composition is effective for treating cancer.

19. A pharmaceutical composition comprising the purified cell composition of claim 10, and a pharmaceutically acceptable carrier.

20. The pharmaceutical composition of claim 19, further comprising venetoclax.

21. A method of treating cancer in a subject in need thereof, comprising administering the pharmaceutical composition of claim 17 to the subject.

22. The method of claim 21, wherein the cancer is AML.

23. The method of claim 21, wherein the cancer is resistant or refractory to a BCL2 inhibitor.

24. The method of claim 23, wherein the BCL2 inhibitor is selected from the group consisting of venetoclax, navitoclax, and obatoclax.

25. The method of claim 23, wherein the BCL2 inhibitor is venetoclax.
